(19) United States
(12) Patent Application Publication
(10) Pub. No.: US 2009/0069179 A1
(43) Pub. Date: Mar. 12, 2009

(54) Pyridin-4-ylmethylamides

(75) Inventors: Jan Klaas Lohmann, Ludwigshafen (DE); Wassilios Grammenos, Ludwigshafen (DE); Michael Puhl, Lampertheim (DE); Jochen Dietz, Mannheim (DE); Bernd Muller, Frankenthal (DE); Joachim Rheinheimer, Ludwigshafen (DE); Jens Renner, Bad Durkheim (DE)

Correspondence Address: BRINKS, HOFER, GILSON & LIONE, P.O. BOX 1340, MORRISVILLE, NC, 27560, US

(73) Assignee: BASF SE, Ludwigshafen (DE)

(21) Appl. No.: 12/279,117

(22) PCT Filed: Feb. 13, 2007
(86) PCT No.: PCT/EP2007/051395

§ 371 Date: Aug. 12, 2008

(30) Foreign Application Priority Data
Feb. 14, 2006 (EP) 06002963.4

(51) Int. Cl. A01C 1/06; C07D 401/12; A01P 3/00; A01N 43/40

(52) U.S. Cl. 504/100; 546/261; 514/335

(57) ABSTRACT

The present invention relates to pyridin-4-ylmethylamides of the general formula (I), where R¹ to R⁶ and n are as defined in the claims and to the N-oxides and the agriculturally acceptable salts of the compounds I. The invention also relates to a process for preparing these compounds. Furthermore, the invention relates to the use of the compounds I and the N-oxides and the agriculturally acceptable salts thereof for combating phytopathogenic fungi (hereinafter referred to as harmful fungi). Furthermore the compounds I, their N-oxides and salts can be used for controlling arthropodal pests.

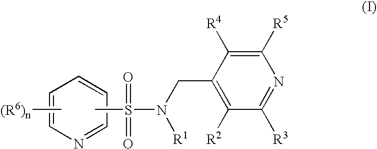

The present invention relates to pyridin-4-ylmethylamides of the general formula I

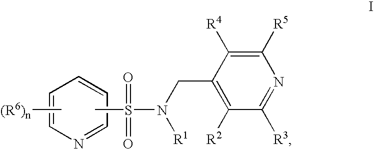

where
R¹ is hydrogen, C₁-C₆-alkyl, C₁-C₆-alkoxy, cyano-C₁-C₄-alkyl, C₁-C₄-haloalkyl, C₁-C₄-alkoxy-C₁-C₄-alkyl, C₁-C₄-haloalkoxy-C₁-C₄-alkyl, di(C₁-C₄-alkyl)amino-C₁-C₄-alkyl, C₃-C₆-cycloalkyl-C₁-C₄-alkyl, C₃-C₆-halocycloalkyl-C₁-C₄-alkyl, (C₁-C₄-alkyl)carbonyl, (C₁-C₄-alkoxy)carbonyl, C₂-C₆-alkenyl, C₃-C₆-cycloalkyl, C₃-C₆-halocycloalkyl, C₅-C₆-cycloalkenyl, saturated 5 or 6-membered N-heterocyclyl-C₁-C₄-alkyl, cyano-C₂-C₄-alkenyl, C₂-C₄-haloalkenyl, C₁-C₄-alkoxy-C₂-C₄-alkenyl, C₁-C₄-haloalkoxy-C₂-C₄-alkenyl, (C₁-C₄-alkyl)carbonyl-C₂-C₄-alkenyl, (C₁-C₄-alkoxy)carbonyl-C₂-C₄-alkenyl, di(C₁-C₄-alkyl)amino-C₂-C₄-alkenyl, C₂-C₆-alkynyl, C₂-C₄-haloalkynyl, C₁-C₄-haloalkyl-C₂-C₄-alkynyl, C₁-C₄-alkoxy-C₂-C₄-alkynyl, tri(C₁-C₄-alkyl)silyl-C₂-C₄-alkynyl, di(C₁-C₄-alkyl)amino, naphthylmethyl or benzyl wherein the last two mentioned radicals may carry at the phenyl or naphthyl ring 1, 2, or 3 radicals, selected from cyano, halogen, C₁-C₄-alkyl, C₁-C₄-haloalkyl, C₁-C₄-alkoxy, C₁-C₄-haloalkoxy, (C₁-C₄-alkyl)carbonyl, (C₁-C₄-alkoxy)carbonyl and di(C₁-C₄-alkyl)amino radical;
R², R³, R⁴, R⁵ independently of one another are selected from hydrogen, halogen, C₁-C₄-alkyl, C₂-C₄-alkenyl, C₂-C₄-alkynyl, tri-C₁-C₄-alkylsilyl, C₁-C₄-haloalkyl, C₁-C₄-alkoxy, C₁-C₄-haloalkoxy, S(O)ₚR¹⁶ and NR¹⁷R¹⁸;
   or
   R² and R³ together with the carbon atoms to which they are attached, may form a fused 5 or 6-membered carbocycle or a fused 5- or 6-membered heterocycle containing one, two or three heteroatoms as ring members, being selected from the group consisting of nitrogen, oxygen and sulfur atoms, it being possible for the fused ring to carry one or two radicals R⁷ and/or R⁸,
   R⁶ is halogen, cyano, nitro, C₁-C₁₀-alkyl, C₂-C₁₀-alkenyl, C₂-C₁₀-alkynyl, C₁-C₁₀-alkoxy, C₁-C₁₀-haloalkyl, C₁-C₁₀-haloalkoxy, (C₁-C₄-alkyl)carbonyl, (C₁-C₄-alkoxy)carbonyl, —C(R⁹)═NOR¹⁰, (C₁-C₄-alkyl)aminocarbonyl, di(C₁-C₄-alkyl)aminocarbonyl, 5- or 6-membered hetaryl or hetaryloxy containing one or two heteroatoms as ring members, being selected from the group of nitrogen, oxygen and sulfur atoms, phenyl, or phenoxy, where the phenyl or hetaryl ring in the last four mentioned radicals may carry one, two or three radicals R¹¹;
   two radicals R⁶ together with two adjacent carbon atoms of the pyridyl ring to which they are attached may also form a fused 5- or 6-membered carbocycle which may be substituted by 1, 2 or 3 radicals R¹²;
R⁷, R⁸ independently of one another are halogen, C₁-C₄-alkyl, C₁-C₄-haloalkyl, C₁-C₄-alkoxy or C₁-C₄-haloalkoxy;
n is 0, or 2;
R⁹ is hydrogen, C₁-C₄-alkyl, C₁-C₄-haloalkyl, C₁-C₄-alkoxy-C₁-C₄-alkyl, C₁-C₄-haloalkoxy-C₁-C₄-alkyl, phenyl which may bear a cyano, halogen, C₁-C₄-alkoxy or C₁-C₄-haloalkoxy radical, or benzyl which may be unsubstituted or substituted with 1, 2 or 3 radicals, selected from cyano, halogen and C₁-C₄-alkyl;
R¹⁰ is C₁-C₆-alkyl, benzyl, C₂-C₄-alkenyl, C₁-C₄-haloalkyl, C₂-C₄-haloalkenyl, C₂-C₄-alkynyl or C₂-C₄-haloalkynyl;
R¹¹ is nitro, cyano, OH, halogen, C₁-C₄-alkyl, C₁-C₄-haloalkyl, C₁-C₄-alkoxy, C₁-C₄-haloalkoxy, (C₁-C₄-alkoxy)carbonyl, C₁-C₄-alkylcarbonyl, CHO, CO—NH₂, C₁-C₄-alkylaminocarbonyl, di(C₁-C₄-alkyl)aminocarbonyl, C₁-C₄-alkylthio, C₁-C₄-haloalkylthio, C₁-C₄-alkylsulfinyl, C₁-C₄-haloalkylsulfinyl, C₁-C₄-alkylsulfonyl, C₁-C₄-haloalkylsulfonyl, (C₁-C₄-alkyl)amino, di(C₁-C₄-alkyl)amino, tri(C₁-C₄-alkyl)silyl, —C(R¹³)═NOR¹⁴, C₂-C₄-alkenyl or C₂-C₄-alkynyl;
   two radicals R¹¹ together with two adjacent carbon atoms of the phenyl ring to which they are attached may form a fused 5- or 6-membered carbocycle or a fused 5- or 6-membered heterocycle containing one, two or three heteroatoms as ring members, being selected from the group consisting of nitrogen, oxygen and sulfur atoms, it being possible for the fused ring to carry 1, 2 or 3 radicals R^{12a};
R¹², R^{12a} independently of each other are selected from halogen, cyano, nitro, C₁-C₈-alkyl, C₁-C₈-haloalkyl, C₁-C₈-alkoxy, C₁-C₈-haloalkoxy, (C₁-C₄-alkyl)carbonyl, (C₁-C₄-alkoxy)carbonyl, —C(R^{13a})═NOR^{14a}, (C₁-C₄-alkyl)aminocarbonyl, di(C₁-C₄-alkyl)aminocarbonyl, phenyl and phenoxy, where the ring in the last two mentioned radicals may carry one, two or three groups R¹⁵;
R¹³, R^{13a} independently of each other are selected from hydrogen, C₁-C₄-alkyl, C₁-C₄-haloalkyl, C₁-C₄-alkoxy-C₁-C₄-alkyl, C₁-C₄-haloalkoxy-C₁-C₄-alkyl, phenyl which may be unsubstituted or substituted with 1, 2 or 3 radicals, selected from cyano, halogen, C₁-C₄-alkoxy and C₁-C₄-haloalkoxy, or benzyl which may be unsubstituted or substituted with 1, 2 or 3 radicals, selected from cyano, halogen and C₁-C₄-alkyl;
R¹⁴, R^{14a} independently of each other are selected from C₁-C₆-alkyl, benzyl, C₂-C₄-alkenyl, C₁-C₄-haloalkyl, C₂-C₄-haloalkenyl, C₂-C₄-alkynyl and C₂-C₄-haloalkynyl;
R¹⁵ is halogen, C₁-C₄-alkyl, C₁-C₄-alkoxy, C₁₋haloalkyl or C₁₋haloalkoxy;
R¹⁶ is C₁-C₄-alkyl or C₁-C₄-haloalkyl and p is 0, 1 or 2; and
R¹⁷, R¹⁸ independently of each other are selected from hydrogen, C₁-C₆-alkyl or R¹⁷ and R¹⁸ together with the nitrogen atom to which they are attached form a five- to eight-membered saturated heterocycle which is attached via nitrogen and may contain one, two or three further heteroatoms or heteroatom groups from the group consisting of O, N, S, S(O) and S(O)₂ as ring members, it being possible for the heterocycle to carry 1, 2, 3 or 4 substituents selected from C₁-C₄-alkyl, C₁-C₄-haloalkyl or halogen; and the N-oxides and the agriculturally acceptable salts of the compounds I.

WO 2005/33081 describes 4-pyridylmethyl amides of benzenesulfonic acid compounds and their use for combating harmful fungi. However, the action of the compounds disclosed there is not always completely satisfying. Therefore, it was an object of the present invention to provide compounds having improved action and/or a broadened activity spectrum against harmful fungi.

It was found that this object is achieved by the compounds of the general formula I, their N-oxides and salts, as defined herein. Compared to the known compounds, the compounds of the formula I have increased efficacy against harmful fungi. Therefore the invention relates to compounds of the general formula I, their N-oxides and the salts thereof. The invention also relates to a process for preparing these compounds.

Furthermore, the invention relates to the use of the compounds I and the N-oxides and the agriculturally acceptable salts thereof for combating phytopathogenic fungi (hereinafter referred to as harmful fungi). Accordingly, the invention also provides a method for combating phytopathogenic fungi which method comprises treating the fungi or the materials, plants, the soil or seeds to be protected against fungal attack, with an effective amount of at least one pyridin-4-ylmethyl-amide of the formula I and/or an N-oxide or an agriculturally acceptable salt thereof.

Accordingly, the invention further provides agricultural compositions, preferably in the form of directly sprayable solutions, emulsions, pastes, oil dispersions, powders, materials for scattering, dusts or in the form of granules, which comprises a pesticidally effective amount of at least one compound I, and/or an N-oxide or a salt thereof, and at least one carrier which may be liquid and/or solid and which is preferably agronomically acceptable, and/or at least one surfactant.

Furthermore, it was found that the compounds I, their N-oxides and salts can be used for controlling or combating arthropodal pests. The compounds I, their N-oxides and salts are in particular useful for combating insects. Likewise the compounds I, their N-oxides and salts are in particular useful for combating arachnids. The term “combating arthropodal pest” as used herein comprises controlling, i.e. killing, of said pests and also protecting plants, non-living materials or seed from an attack or infestation by said pests. Therefore the invention relates to the use of the compounds I and the N-oxides and the agriculturally acceptable salts thereof, for combating arthropodal pests.

Furthermore, the invention provides a method for combating such pests, which comprises contacting said pests, their habitat, breeding ground, food supply, plant, seed, soil, area, material or environment in which the arthropodal pests are growing or may grow, or the materials, plants, seeds, soils, surfaces or spaces to be protected from an attack of or infestation by said pest, with a pesticidally effective amount of at least one pyridin-4-ylmethyl-amid compound of the formula I, and/or an N-oxide or salt thereof, or with a composition comprising at least one pyridin-4-ylmethyl-amid compound of the formula I, and/or N-oxide, or agriculturally acceptable salt thereof, as defined herein.

The invention provides in particular a method for protecting crops, including seeds, from attack or infestation by arthropodal pests and/or infection by phytopathogenic fungi, said method comprises contacting a crop with an effective amount of at least one compound of formula I and/or the N-oxide or salt thereof, as defined herein. The invention also provides seeds, comprising at least one pyridin-4-ylmethyl-amide compound of the formula I, and/or an N-oxide or an agriculturally acceptable salt thereof, preferably in an amount of from 0.1 g to 10 kg per 100 kg of seed.

The invention also provides a method for protecting non-living materials from attack or infestation by the aforementioned pests and/or harmful fungi, which method comprises contacting the non-living material with a pesticidally effective amount of at least one compound of formula I as defined herein, with an N-oxide thereof or with a salt thereof.

Suitable compounds of formula I encompass all possible stereoisomers (cis/trans isomers, enantiomers) which may occur and mixtures thereof. Stereoisomeric centers are e.g. the carbon and nitrogen atom of the —C(R⁹)═NOR¹⁰ moiety as well as asymmetric carbon atoms in the radicals R¹, R², R³, R⁴ and/or R⁵ etc. The present invention provides both the pure enantiomers or diastereomers or mixtures thereof, the pure cis- and trans-isomers and the mixtures thereof. The compounds of the general formula I may also exist in the form of different tautomers. The invention comprises the single tautomers, if separable, as well as the tautomer mixtures. The present invention includes both the (R)- and (S)-isomers of compounds of the formula I having chiral centers as well as mixtures thereof, in particular the racemates thereof.

Salts of the compounds of the formula I and of the N-oxides of formula I are agriculturally acceptable salts. They can be formed by customary methods, e.g. by reacting the compound with an acid of the anion in question if the compound of formula I has a basic functionality or by reacting an acidic compound of formula I with a suitable base.

Suitable agriculturally useful salts include the salts of those cations or the acid addition salts of those acids whose cations and anions, respectively, do not have any adverse effect on the action of the compounds according to the present invention. Suitable cations are in particular the ions of the alkali metals, preferably lithium, sodium and potassium, of the alkaline earth metals, preferably calcium, magnesium and barium, and of the transition metals, preferably manganese, copper, zinc and iron, and also ammonium (NH₄⁺) and substituted ammonium in which one to four of the hydrogen atoms are replaced by C₁-C₄-alkyl, C₁-C₄-hydroxyalkyl, C₁-C₄-alkoxy, C₁-C₄-alkoxy-C₁-C₄-alkyl, hydroxy-C₁-C₄-alkoxy-C₁-C₄-alkyl, phenyl or benzyl. Examples of substituted ammonium ions comprise methylammonium, isopropylammonium, dimethylammonium, diisopropylammonium, trimethylammonium, tetramethylammonium, tetraethylammonium, tetrabutylammonium, 2-hydroxyethylammonium, 2-(2-hydroxyethoxy)ethylammonium, bis(2-hydroxyethyl)ammonium, benzyltrimethylammonium and benzyltriethylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogen sulfate, sulfate, dihydrogen phosphate, hydrogen phosphate, phosphate, nitrate, hydrogen carbonate, carbonate, hexafluorosilicate, hexafluorophosphate, benzoate, and the anions of C₁-C₄-alkanoic acids, preferably formate, acetate, propionate and butyrate. They can be formed by reacting the compounds of formula I with an acid of the corresponding anion, preferably of hydrochloric acid, hydrobromic acid, sulfuric acid, phosphoric acid or nitric acid.

The organic moieties mentioned in the above definitions of the variables are—like the term halogen—collective terms for individual listings of the individual group members. The prefix Cₙ-Cₘ indicates in each case the possible number of carbon atoms in the group.

halogen: fluorine, chlorine, bromine and iodine;
alkyl and all alkyl moieties in alkylcarbonyl, tri(alkyl)silyl, dialkylamino, dialkylaminocarbonyl: saturated straight-chain or branched hydrocarbon radicals having 1 to 4, 6, 8 or 10 carbon atoms, preferably 1 to 6 carbon atoms (C₁-C₆-alkyl), especially 1 to 4 carbon atoms (C₁-C₄-alkyl) such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; alkyl having 1 to 10 carbon atoms (C₁-C₁₀-alkyl): C₁-C₆-alkyl as mentioned above, and also, for example heptyl, octyl, 2-ethylhexyl, 2,4,4-trimethylpentyl, 1,1,3,3-tetramethylbutyl, nonyl and decyl;
alkoxy: saturated straight-chain or branched hydrocarbon radicals having 1 to 4, 6, 8 or 10 carbon atoms, preferably 1 to 6 carbon atoms, especially 1 to 4 carbon atoms, as defined herein, which is attached to the remainder of the molecule via an oxygen linkage;
haloalkyl: straight-chain or branched alkyl groups having 1 to 2, 4, 6, 8 or 10 carbon atoms (as mentioned above), where some or all of the hydrogen atoms in these groups may be replaced by halogen atoms as mentioned above: in particular C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl or 1,1,1-trifluoroprop-2-yl;
haloalkoxy and all haloalkoxy moieties in haloalkoxyalkyl, haloalkoxyalkenyl: straight-chain or branched alkyl groups having 1 to 4, 6, 8 or 10 carbon atoms, in particular 1 to 6 carbon atoms (C₁-C₆-haloalkyl), especially 1 to 4 carbon atom (C₁-C₄-haloalkyl), as mentioned above bonded through oxygen linkage, at any bond in the alkyl group, where some or all of the hydrogen atoms in these groups may be replaced by halogen atoms as mentioned above, for example C₁-C₂-haloalkoxy, such as chloromethoxy, bromomethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 1-chloroethoxy, 1-bromoethoxy, 1-fluoroethoxy, 2-fluoroethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, 5-fluoropentoxy, 5-chloropentoxy, 5-bromopentoxy, 5-iodopentoxy, 6-fluorohexoxy, 6-chlorohexoxy, 6-bromohexoxy or 6-iodohexoxy and the like;
haloalkylthio: straight-chain or branched alkyl group having 1 to 4 carbon atoms, as mentioned above which is attached to the remainder of the molecule via a sulfur linkage, where some or all of the hydrogen atoms may be replaced by halogen atoms as mentioned above;
haloalkylsulfinyl: straight-chain or branched alkyl group having 1 to 4 carbon atoms, as mentioned above which is attached to the remainder of the molecule via an SO group, where some or all of the hydrogen atoms may be replaced by halogen atoms as mentioned above;
haloalkylsulfonyl: straight-chain or branched alkyl group having 1 to 4 carbon atoms, as mentioned above which is attached to the remainder of the molecule via an SO₂ group, where some or all of the hydrogen atoms may be replaced by halogen atoms as mentioned above;
alkenyl: unsaturated straight-chain or branched hydrocarbon radicals having 2 to 4, 6, 8 or 10 carbon atoms and one or two double bonds in any position, for example C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;
haloalkenyl: unsaturated straight-chain or branched hydrocarbon radicals having 2 to 4 carbon atoms and one or two double bonds in any position (as mentioned above), where in these groups some or all of the hydrogen atoms may be replaced by halogen atoms as mentioned above, in particular by fluorine, chlorine and bromine;
alkynyl: straight-chain or branched hydrocarbon groups having 2 to 4, 6, 8 or 10 carbon atoms and one or two triple bonds in any position, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;
haloalkynyl: unsaturated straight-chain or branched hydrocarbon radicals having 2 to 4 carbon atoms and one triple bond in any position (as mentioned above), where in these groups some or all of the hydrogen atoms may be replaced by halogen atoms as mentioned above, in particular by fluorine, chlorine and bromine;
cycloalkyl: mono- or bicyclic saturated hydrocarbon groups having 3 to 6 carbon ring members, for example C₃-C₆-cycloalkyl such as cyclopropyl, cyclobutyl, cyclopentyl, and cyclohexyl;
cycloalkenyl: monocyclic monounsaturated hydrocarbon groups having 5 to 6 carbon ring members (C₅-C₆-cycloalkenyl), such as cyclopenten-1-yl, cyclopenten-3-yl, cyclohexen-1-yl, cyclohexen-3-yl and cyclohexen-4-yl;
tri(C₁-C₄-alkyl)silyl: silicium radical carrying 3 C₁-C₄-alkyl groups, which may be identical or different, examples including trimethylsilyl, triethylsilyl, dimethylethylsilyl, dimethylisopropylsilyl, dimethyl-n-butylsilyl, dimethyl-2-butylsilyl, etc.;
the terms “cyano-C₁-C₄-alkyl”, “C₁-C₄-alkoxy-C₁-C₄-alkyl”, “C₁-C₄-haloalkoxy-C₁-C₄-alkyl”, “di(C₁-C₄-alkyl)amino-C₁-C₄-alkyl”, “C₃-C₆-cycloalkyl-C₁-C₄-alkyl”, “C₃-C₆-halocycloalkyl-C₁-C₄-alkyl”, “saturated 5 or 6-membered N-heterocyclyl-C₁-C₄-alkyl”, as used herein, refer to C₁-C₄-alkyl, as defined herein, which is substituted by one radical selected from cyano, C₁-C₄-alkoxy, C₁-C₄-haloalkoxy, di(C₁-C₄-alkyl)amino, C₃-C₆-cycloalkyl, C₃-C₆-halocycloalkyl, saturated 5 or 6-membered N-heterocyclyl;
the terms “cyano-C₂-C₄-alkenyl”, “C₁-C₄-alkoxy-C₂-C₄-alkenyl”, “C₁-C₄-haloalkoxy-C₂-C₄-alkenyl”, “(C₁-C₄-alkyl)carbonyl-C₂-C₄-alkenyl”, “(C₁-C₄-alkoxy)carbonyl-C₂-C₄-alkenyl”, “di(C₁-C₄-alkyl)amino-C₂-C₄-alkenyl” refer to C₂-C₄-alkenyl, as defined herein,
which is substituted by one radical selected from cyano, C₁-C₄-alkoxy, C₁-C₄-haloalkoxy, (C₁-C₄-alkyl)carbonyl, (C₁-C₄-alkoxy)carbonyl, di(C₁-C₄-alkyl)amino; the terms “C₁-C₄-haloalkyl-C₂-C₄-alkynyl”, “C₁-C₄-alkoxy-C₂-C₄-alkynyl”, “tri(C₁-C₄-alkyl)silyl-C₂-C₄-alkynyl” refer to C₂-C₄-alkynyl, as defined herein, which is substituted by one radical selected from C₁-C₄-haloalkyl, C₁-C₄-alkoxy, tri(C₁-C₄-alkyl)silyl;
five- or six-membered heterocycle which contains one, two, three or four heteroatoms from the group consisting of O, N and S, is to be understood as meaning both saturated, partially unsaturated and aromatic heterocycles having 5 or 6 ring atoms, including:
5- or 6-membered heterocyclyl which contains one, two or three nitrogen atoms and/or one oxygen or sulfur atom or one or two oxygen and/or sulfur atoms, and which is saturated or partially unsaturated, for example 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 2-pyrrolin-2-yl, 2-pyrrolin-3-yl, 3-pyrrolin-2-yl, 3-pyrrolin-3-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl and 2-piperazinyl;
5-membered aromatic heterocyclyl (heteroaryl) which contains one, two, three or four nitrogen atoms or one, two or three nitrogen atoms and one sulfur or oxygen atom: 5-membered heteroaryl groups which, in addition to carbon atoms, may contain one to four nitrogen atoms or one to three nitrogen atoms and one sulfur or oxygen atom as ring members, for example 2-thienyl, 3-thienyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl and 1,3,4-triazol-2-yl;
6-membered heteroaryl which contains one, two, three or four nitrogen atoms: 6-membered heteroaryl groups which, in addition to carbon atoms, may contain one, two, three or four nitrogen atoms as ring members, for example 2-pyridinyl, 3-pyridinyl, 4-pyridinyl, 3-pyridazinyl, 4-pyridazinyl, 2-pyrimidinyl, 4-pyrimidinyl, 5-pyrimidinyl and 2-pyrazinyl.

Likewise a five- to eight-membered saturated heterocycle which is attached via nitrogen and may contain one, two or three further heteroatoms or heteroatom groups from the group consisting of O, N, S, S(O) and S(O)₂ as ring members, is a saturated heterocycle, which contains a nitrogen atom as ring member and which is bound to the remainder of the molecule via said nitrogen atom, and which has 5, 6, 7 or 8 ring atoms which are carbon atoms or heteroatoms such as O, N or S or heteroatom groups such as S(O) or S(O)₂; examples including pyrrolidin-1-yl, piperazin-1-yl, morpholin-4-yl, thiomorpholin-4-yl, azepan-1-yl etc.

Fused 5- or 6-membered carbocycle means a hydrocarbon ring which shares two adjacent carbon atoms with another ring, examples being cyclopentane, cyclopentene, cyclohexane, cyclohexene and benzene.

Examples for 5- or 6-membered heterocycles which contain a contain a fused 5- or 6-membered carbocyclic ring as mentioned above are indolyl, indolinyl, isoindolinyl, benzpyrazolyl, benzimidazolyl, benzotriazolyl, quinolinyl, 1,2,3,4-tetrahydroquinolinyl, isoquinolinyl, phthalazinyl, quinazinyl, quinazolinyl, cinnolinyl, benzofuranyl, benzothiophenyl, benzopyranyl, dihydrobenzopyranyl, benzothiopyranyl, 1,3-benzodioxolyl, benzoxazolyl, benzthiazolyl, benzisoxazolyl and 1,4-benzodioxanyl.

Alkylene: divalent unbranched chains of 1 to 5 CH₂ groups, for example CH₂, CH₂CH₂, CH₂CH₂CH₂, CH₂CH₂CH₂CH₂ and CH₂CH₂CH₂CH₂CH₂;

Alkenylen: divalent unbranched chains of 4 or 6 CH groups which are linked by conjugated C═C double bonds, for example CH═CH or CH═CH—CH═CH.

With a view to the intended uses of the pyridin-4-ylmethyl-amides I, particular preference is given to the following meanings of the substituents, in each case on their own or in combination:

The invention preferably provides compounds of the formula I in which R¹ is hydrogen, C₁-C₄-alkyl, C₃-C₄-alkenyl such as allyl, C₃-C₄-alkynyl such as propargyl, or benzyl, in particular hydrogen.

Preference is also given to compounds of the formula I in which R², R³, R⁴ and R⁵ independently of one another are selected from hydrogen, halogen, C₁-C₄-alkyl, C₂-C₄-alkenyl, C₂-C₄-alkynyl, tri-C₁-C₄-alkylsilyl, C₁-C₄-haloalkyl, C₁-C₄-alkoxy, C₁-C₄-haloalkoxy, S(O)ₚR¹⁶ and NR¹⁷R¹⁸, in particular hydrogen, C₁-C₄-alkyl such as methyl or ethyl, halogen, such as fluorine or chlorine, C₁-C₂-haloalkyl such as CF₃, or C₁-C₂-haloalkoxy such as OCF₃ or OCHF₂.

Particular preference is given to compounds wherein R², R³, R⁴ and R⁵ are hydrogen.

In addition, particular preference is also given to compounds of the formula I in which at least one, in particular one or two, group(s) selected from R², R³, R⁴ and R⁵ is/are not hydrogen. Amongst these, preference is given to those compounds, wherein both R⁴ and R⁵ are hydrogen, while at least one of the radicals R², R³ is different from hydrogen and has one of the meanings given above. In particular the R² and/or R³ that is different from hydrogen is selected from C₁-C₄-alkyl such as methyl or ethyl, halogen, such as fluorine or chlorine, C₁-C₂-haloalkyl such as CF₃, or C₁-C₂-haloalkoxy such as OCF₃ or OCHF₂. In this embodiment preference is also given to compounds, wherein one of the radicals R² and/or R³ is selected from C₂-C₄-alkenyl, C₂-C₄-alkynyl, tri(C₁-C₄-alkyl)silyl, a radical S(O)ₚR¹⁶ or a radical NR¹⁷R¹⁸. The remaining radical R² or R³ is preferably hydrogen or selected from the group consisting of C₁-C₄-alkyl such as methyl or ethyl, halogen, such as fluorine or chlorine, C₁-C₂-haloalkyl such as CF₃, or C₁-C₂-haloalkoxy such as OCF₃ or OCHF₂.

Preference is likewise also given to compounds of the formula I, wherein the radicals R² and R³ together with the atoms to which they are bound form a fused benzene ring, i.e. R² and R³ together form a bivalent radical —CH═CH—CH═CH—, wherein one or two of the hydrogen atoms may be replaced by the radicals R⁷ and/or R⁸. In this embodiment, R⁴ and R⁵ are preferably hydrogen.

Preferably n is 1 or 2. If n is 1 or 2, preferably at least one radical R⁶ is located meta or para with respect to the sulfonyl group.

In a first preferred embodiment, n is 1 or 2 and R⁶ is selected from halogen, in particular chlorine and fluorine; C₁-C₄-alkyl, in particular methyl and ethyl; C₁-C₄-alkoxy, in particular methoxy and ethoxy; C₁-C₄-haloalkyl, in particular trifluoromethyl; C₁-C₄-haloalkoxy, in particular difluoromethoxy and trifluoromethoxy; (C₁-C₄-alkoxy)carbonyl, in particular methoxycarbonyl and ethoxycarbonyl.

In a second preferred embodiment n is 1 or 2 and one of the radicals R⁶ is phenyl or 5- or 6-membered hetaryl, which are unsubstituted or preferably carry 1, 2 or 3 radicals R¹¹ as defined above.

More preference is given to compounds wherein one of the radicals R⁶ is phenyl, which is unsubstituted or which preferably carries 1, 2 or 3 radicals R¹¹ as defined above. If present, the further radical R⁶ is preferably different from phenyl, hetaryl, hetaryloxy or phenoxy, and more preferably selected from halogen, in particular chlorine and fluorine; C₁-C₄-alkyl, in particular methyl and ethyl; C₁-C₄-alkoxy, in particular methoxy and ethoxy; C₁-C₄-haloalkyl, in particular trifluoromethyl; C₁-C₄-haloalkoxy, in particular difluoromethoxy and trifluoromethoxy; (C₁-C₄-alkoxy)carbonyl, in particular methoxycarbonyl and ethoxycarbonyl.

In the second embodiment n is preferably 1. In the second embodiment, the phenyl ring or the hetaryl ring is preferably located meta or para with respect to the sulfonyl group.

Likewise preferred are compounds of the formula I, wherein R⁶ is 5- or 6-membered hetaryl or hetaryloxy containing one or two heteroatoms as ring members, selected from the group of nitrogen, oxygen and sulfur atoms, wherein the heterocycle may be unsubstituted or may carry 1, 2 or 3 radicals R¹¹. In this preferred embodiment R⁶ is preferably 5- or 6-membered hetaryl, in particular, pyrdiyl, thienyl, oxazolyl, isoxyzolyl, oxadiazolyl or thiadizolyl, more preferably 2-, 3- or 4-pyridyl, oxazol-5-yl, oxazol-2-yl or 1,3,4-oxadiazol-2-yl, wherein the hetaryl may be unsubstituted or may carry 1, 2 or 3 more preferably 1 or 2 radicals R¹¹ as defined herein.

In a further preferred embodiment of the compounds I according to the invention, the index n is zero.

In the compounds of the formula I, the pyridine ring at the sulfonyl group may be bound via the carbon atom in the 2-, 3- or 4-position of the pyridine ring, i.e. the nitrogen atom of the pyridine ring may be located ortho, meta or para with respect to the sulfonyl group.

Consequently, one embodiment of the invention relates to compounds of the formula I-A,

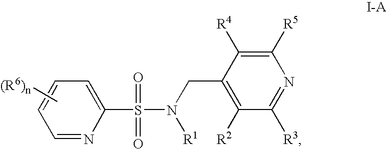

wherein R¹, R², R³, R⁴, R⁵, R⁶ and n are as defined herein. Amongst compounds I-A, preference is given to those, wherein n is 1 or 2 and wherein one radical R⁶ is located in the 6-position of the pyridine ring. These compounds are also referred to as compounds I-A a. Preference is also given to compounds I-A, wherein n is 1 or 2, in particular 1, and wherein one radical R⁶ is located in the 5-position of the pyridine ring. These compounds are also referred to as compounds I-A.b. Preference is also given to compounds I-A, wherein n is 1 or 2, in particular 1, and wherein one radical R⁶ is located in the 4-position of the pyridine ring. These compounds are also referred to as compounds I-A.c. In the compounds I-A a, I-A.b and I-A.c the radical R⁶, which is located in the 4-, 5- or 6-position, is most preferably phenyl, which is unsubstituted or substituted as defined above.

Consequently, a further embodiment of the invention relates to compounds of the formula I-B,

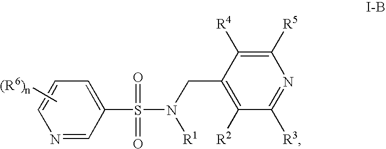

wherein R¹, R², R³, R⁴, R⁵, R⁶ and n are as defined herein. Amongst compounds I-B, preference is given to those, wherein n is 1 or 2 and one radical R⁶ is located in the 6-position of the pyridine ring. These compounds are also referred to as compounds I-B.a. Preference is also given to compounds I-B, wherein n is 1 or 2 and one radical R⁶ is located in the 5-position of the pyridine ring. These compounds are also referred to as compounds I-B.b. In the compounds I-B.a, and I-B.b the radical R⁶, which is located in the 5- or 6-position, is most preferably phenyl, which is unsubstituted or substituted as defined above.

Consequently, a further embodiment of the invention relates to compounds of the formula I-C,

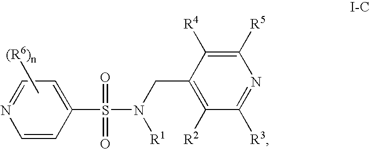

wherein R¹, R², R³, R⁴, R⁵, R⁶ and n are as defined herein. Amongst compounds I-C, preference is given to those, wherein n is 1 or 2 and one radical R⁶ is located in the 2-position of the pyridine ring. These compounds are also referred to as compounds I-C.a. In the compounds I-C.a, the radical R⁶, which is located in the 2-position, is most preferably phenyl, which is unsubstituted or substituted as defined above.

R⁷, if present, is preferably selected from halogen, in particular chlorine and fluorine; C₁-C₄-alkyl, in particular methyl, ethyl, isopropyl, tert.-butyl; C₁-C₄-alkoxy, in particular methoxy, ethoxy, isopropoxy, tert.-butoxy; and C₁-C₄-haloalkyl, in particular trifluoromethyl and pentafluoroethyl.

R⁸, if present, is preferably selected from halogen, in particular chlorine and fluorine; C₁-C₄-alkyl, in particular methyl, ethyl, isopropyl, tert.-butyl; C₁-C₄-alkoxy, in particular methoxy, ethoxy, isopropoxy, tert.-butoxy; and C₁-C₄-haloalkyl, in particular trifluoromethyl and pentafluoroethyl.

R⁹, R¹³, R^{13a}, if present, are independently of each other preferably selected from hydrogen or C₁-C₄-alkyl, in particular hydrogen.

R¹⁰, R¹⁴, R^{14a}, if present, are independently of each other preferably C₁-C₄-alkyl.

R¹¹, if present, is preferably selected from nitro, CN, OH, halogen, C₁-C₄-alkyl, C₁-C₄-haloalkyl, C₁-C₄-alkoxy, C₁-C₄-haloalkoxy, (C₁-C₄-alkoxy)carbonyl, C₁-C₄-alkylcarbonyl, C₁-C₄-alkylthio, C₁-C₄-haloalkylthio, C₁-C₄-alkylsulfonyl, C₁-C₄-haloalkylsulfonyl, (C₁-C₄-alkyl)amino, di(C₁-C₄-alkyl)amino, tri(C₁-C₄-alkyl)silyl, —CH═NO(C₁-C₄-alkyl), —C(C₁-C₄-alkyl)═NO(C₁-C₄-alkyl), C₂-C₄-alkenyl, C₃-C₄-alkynyl or CONH₂, or two radicals R¹¹ together with two adjacent carbon atoms of the phenyl ring may form a radical of the formulae: (CH₂)₃, (CH₂)₄, O—CH₂—O, O(CH₂)₃ or —CH═CH—CH═CH—. R¹¹, if present, is more preferably selected from CN, halogen, in particular fluorine or chlorine, C₁-C₄-alkyl, in particular methyl, ethyl, n-propyl, isopropyl or tert.-butyl, C₁-C₄-haloalkyl, in particular trifluoromethyl, difluoromethyl or trifluoroethyl, C₁-C₄-alkoxy, in particular methoxy, C₁-C₄-haloalkoxy, in particular trifluoromethoxy, C₁-C₄-alkylcarbonyl, in particular acetyl, CONH₂, —CH═NOCH₃, —C(CH₃)═NOCH₃, —CH═NOCH₂CH₃, or —C(CH₃)═NOCH₂CH₃.

R¹⁶, if present, is preferably selected from methyl, ethyl, trifluoromethyl, 2-fluoroethyl, 2,2-difluoroethyl or 2,2,2-trifluoroethyl.

The radical NR¹⁷R¹⁸, if present, is preferably selected from NH₂, methylamino, dimethylamino, ethylamino, diethylamino, propylamino, propylmethylamino, dipropylamino, 1-pyrrolidinyl, 1-piperidinyl, 1-piperazinyl, 4-methylpiperazin-1-yl, morpholin-4-yl, 2-methylmorpholin-4-yl or 2,6-dimethylmorpholin-4-yl.

Most preferably R⁶ is phenyl which carries one, two or three radicals R¹¹ as defined herein, in particular as given in the lines of table A. In table A, the prefix indicates the position of the phenyl ring, to which the radical R¹¹ is bound.

Examples of preferred compounds are given in the following tables:

### Table 1

Compounds of the formula I-A.a in which R¹, R², R³, R⁴ and R⁵ are hydrogen, n is 1 and R⁶ is a phenyl ring, which is located in the 6-position of the pyridine ring and which carries 1 or 2 radicals R¹¹ as defined in the rows of Table A;

### Table 2

Compounds of the formula I-A.a in which R¹, R⁴ and R⁵ are hydrogen, R² is hydrogen, R³ is chlorine, n is 1 and R⁶ is a phenyl ring, which is located in the 6-position of the pyridine ring and which carries 1 or 2 radicals R¹¹ as defined in the rows of Table A;

### Table 3

Compounds of the formula I-A.a in which R¹, R⁴ and R⁵ are hydrogen, R² is chlorine, R³ is hydrogen, n is 1 and R⁶ is a phenyl ring, which is located in the 6-position of the pyridine ring and which carries 1 or 2 radicals R¹¹ as defined in the rows of Table A;

### Table 4

Compounds of the formula I-A.a in which R¹, R⁴ and R⁵ are hydrogen, R² is chlorine, R³ is chlorine, n is 1 and R⁶ is a phenyl ring, which is located in the 6-position of the pyridine ring and which carries 1 or 2 radicals R¹¹ as defined in the rows of Table A;

### Table 5

Compounds of the formula I-A.a in which R¹, R⁴ and R⁵ are hydrogen, R² is methoxy, R³ is methyl, n is 1 and R⁶ is a phenyl ring, which is located in the 6-position of the pyridine ring and which carries 1 or 2 radicals R¹¹ as defined in the rows of Table A;

### Table 6

Compounds of the formula I-A.a in which R¹, R⁴ and R⁵ are hydrogen, R² is methyl, R³ is methoxy, n is 1 and R⁶ is a phenyl ring, which is located in the 6-position of the pyridine ring and which carries 1 or 2 radicals R¹¹ as defined in the rows of Table A;

### Table 7

Compounds of the formula I-A.a in which R¹, R⁴ and R⁵ are hydrogen, R² is hydrogen, R³ is methoxy, n is 1 and R⁶ is a phenyl ring, which is located in the 6-position of the pyridine ring and which carries 1 or 2 radicals R¹¹ as defined in the rows of Table A;

### Table 8

Compounds of the formula I-A.a in which R¹, R⁴ and R⁵ are hydrogen, R² is methoxy, R³ is hydrogen, n is 1 and R⁶ is a phenyl ring, which is located in the 6-position of the pyridine ring and which carries 1 or 2 radicals R¹¹ as defined in the rows of Table A;

### Table 9

Compounds of the formula I-A.a in which R¹, R⁴ and R⁵ are hydrogen, R² is methoxy, R³ is methoxy, n is 1 and R⁶ is a phenyl ring, which is located in the 6-position of the pyridine ring and which carries 1 or 2 radicals R¹¹ as defined in the rows of Table A;

### Table 10

Compounds of the formula I-A.a in which R¹, R⁴ and R⁵ are hydrogen, R² is methyl, R³ is hydrogen, n is 1 and R⁶ is a phenyl ring, which is located in the 6-position of the pyridine ring and which carries 1 or 2 radicals R¹¹ as defined in the rows of Table A;

### Table 11

Compounds of the formula I-A.a in which R¹, R⁴ and R⁵ are hydrogen, R² is hydrogen, R³ is methyl, n is 1 and R⁶ is a phenyl ring, which is located in the 6-position of the pyridine ring and which carries 1 or 2 radicals R¹¹ as defined in the rows of Table A;

### Table 12

Compounds of the formula I-A.a in which R¹, R⁴ and R⁵ are hydrogen, R² is methyl, R³ is methyl, n is 1 and R⁶ is a phenyl ring, which is located in the 6-position of the pyridine ring and which carries 1 or 2 radicals R¹¹ as defined in the rows of Table A;

### Table 13

Compounds of the formula I-A.a in which R¹, R⁴ and R⁵ are hydrogen, R² is hydrogen, R³ is OCHF₂, n is 1 and R⁶ is a phenyl ring, which is located in the 6-position of the pyridine ring and which carries 1 or 2 radicals R¹¹ as defined in the rows of Table A;

### Table 14

Compounds of the formula I-A.a in which R¹, R⁴ and R⁵ are hydrogen, R² is OCHF₂, R³ is hydrogen, n is 1 and R⁶ is a phenyl ring, which is located in the 6-position of the pyridine ring and which carries 1 or 2 radicals R¹¹ as defined in the rows of Table A;

### Table 15

Compounds of the formula I-A.b in which R¹, R², R³, R⁴ and R⁵ are hydrogen, n is 1 and R⁶ is a phenyl ring, which is located in the 5-position of the pyridine ring and which carries 1 or 2 radicals R¹¹ as defined in the rows of Table A;

### Table 16

Compounds of the formula I-A.b in which R¹, R⁴ and R⁵ are hydrogen, R² is hydrogen, R³ is chlorine, n is 1 and R⁶ is a phenyl ring, which is located in the 5-position of the pyridine ring and which carries 1 or 2 radicals R¹¹ as defined in the rows of Table A;

### Table 17

Compounds of the formula I-A.b in which R¹, R⁴ and R⁵ are hydrogen, R² is chlorine, R³ is hydrogen, n is 1 and R⁶ is a phenyl ring, which is located in the 5-position of the pyridine ring and which carries 1 or 2 radicals R¹¹ as defined in the rows of Table A;

### Table 18

Compounds of the formula I-A.b in which R¹, R⁴ and R⁵ are hydrogen, R² is chlorine, R³ is chlorine, n is 1 and R⁶ is a phenyl ring, which is located in the 5-position of the pyridine ring and which carries 1 or 2 radicals R¹¹ as defined in the rows of Table A;

### Table 19

Compounds of the formula I-A.b in which R¹, R⁴ and R⁵ are hydrogen, R² is methoxy, R³ is methyl, n is 1 and R⁶ is a phenyl ring, which is located in the 5-position of the pyridine ring and which carries 1 or 2 radicals R¹¹ as defined in the rows of Table A;

### Table 20

Compounds of the formula I-A.b in which R¹, R⁴ and R⁵ are hydrogen, R² is methyl, R³ is methoxy, n is 1 and R⁶ is a phenyl ring, which is located in the 5-position of the pyridine ring and which carries 1 or 2 radicals R¹¹ as defined in the rows of Table A;

### Table 21

Compounds of the formula I-A.b in which R¹, R⁴ and R⁵ are hydrogen, R² is hydrogen, R³ is methoxy, n is 1 and R⁶ is a phenyl ring, which is located in the 5-position of the pyridine ring and which carries 1 or 2 radicals R¹¹ as defined in the rows of Table A;

### Table 22

Compounds of the formula I-A.b in which R¹, R⁴ and R⁵ are hydrogen, R² is methoxy, R³ is hydrogen, n is 1 and R⁶ is a phenyl ring, which is located in the 5-position of the pyridine ring and which carries 1 or 2 radicals R¹¹ as defined in the rows of Table A;

### Table 23

Compounds of the formula I-A.b in which R¹, R⁴ and R⁵ are hydrogen, R² is methoxy, R³ is methoxy, n is 1 and R⁶ is a phenyl ring, which is located in the 5-position of the pyridine ring and which carries 1 or 2 radicals R¹¹ as defined in the rows of Table A;

### Table 24

Compounds of the formula I-A.b in which R¹, R⁴ and R⁵ are hydrogen, R² is methyl, R³ is hydrogen, n is 1 and R⁶ is a phenyl ring, which is located in the 5-position of the pyridine ring and which carries 1 or 2 radicals R¹¹ as defined in the rows of Table A;

### Table 25

Compounds of the formula I-A.b in which R¹, R⁴ and R⁵ are hydrogen, R² is hydrogen, R³ is methyl, n is 1 and R⁶ is a phenyl ring, which is located in the 5-position of the pyridine ring and which carries 1 or 2 radicals R¹¹ as defined in the rows of Table A;

### Table 26

Compounds of the formula I-A.b in which R¹, R⁴ and R⁵ are hydrogen, R² is methyl, R³ is methyl, n is 1 and R⁶ is a phenyl ring, which is located in the 5-position of the pyridine ring and which carries 1 or 2 radicals R¹¹ as defined in the rows of Table A;

### Table 27

Compounds of the formula I-A.b in which R¹, R⁴ and R⁵ are hydrogen, R² is hydrogen, R³ is OCHF₂, n is 1 and R⁶ is a phenyl ring, which is located in the 5-position of the pyridine ring and which carries 1 or 2 radicals R¹¹ as defined in the rows of Table A;

### Table 28

Compounds of the formula I-A.b in which R¹, R⁴ and R⁵ are hydrogen, R² is OCHF₂, R³ is hydrogen, n is 1 and R⁶ is a phenyl ring, which is located in the 5-position of the pyridine ring and which carries 1 or 2 radicals R¹¹ as defined in the rows of Table A;

### Table 29

Compounds of the formula I-A.c in which R¹, R², R³, R⁴ and R⁵ are hydrogen, n is 1 and R⁶ is a phenyl ring, which is located in the 4-position of the pyridine ring and which carries 1 or 2 radicals R¹¹ as defined in the rows of Table A;

### Table 30

Compounds of the formula I-A.c in which R¹, R⁴ and R⁵ are hydrogen, R² is hydrogen, R³ is chlorine, n is 1 and R⁶ is a phenyl ring, which is located in the 4-position of the pyridine ring and which carries 1 or 2 radicals R¹¹ as defined in the rows of Table A;

### Table 31

Compounds of the formula I-A.c in which R¹, R⁴ and R⁵ are hydrogen, R² is chlorine, R³ is hydrogen, n is 1 and R⁶ is a phenyl ring, which is located in the 4-position of the pyridine ring and which carries 1 or 2 radicals R¹¹ as defined in the rows of Table A;

### Table 32

Compounds of the formula I-A.c in which R¹, R⁴ and R⁵ are hydrogen, R² is chlorine, R³ is chlorine, n is 1 and R⁶ is a phenyl ring, which is located in the 4-position of the pyridine ring and which carries 1 or 2 radicals R¹¹ as defined in the rows of Table A;

### Table 33

Compounds of the formula I-A.c in which R¹, R⁴ and R⁵ are hydrogen, R² is methoxy, R³ is methyl, n is 1 and R⁶ is a phenyl ring, which is located in the 4-position of the pyridine ring and which carries 1 or 2 radicals R¹¹ as defined in the rows of Table A;

### Table 34

Compounds of the formula I-A.c in which R¹, R⁴ and R⁵ are hydrogen, R² is methyl, R³ is methoxy, n is 1 and R⁶ is a phenyl ring, which is located in the 4-position of the pyridine ring and which carries 1 or 2 radicals R¹¹ as defined in the rows of Table A.

### Table 35

Compounds of the formula I-A.c in which R¹, R⁴ and R⁵ are hydrogen, R² is hydrogen, R³ is methoxy, n is 1 and R⁶ is a phenyl ring, which is located in the 4-position of the pyridine ring and which carries 1 or 2 radicals R¹¹ as defined in the rows of Table A;

### Table 36

Compounds of the formula I-A.c in which R¹, R⁴ and R⁵ are hydrogen, R² is methoxy, R³ is hydrogen, n is 1 and R⁶ is a phenyl ring, which is located in the 4-position of the pyridine ring and which carries 1 or 2 radicals R¹¹ as defined in the rows of Table A;

### Table 37

Compounds of the formula I-A.c in which R¹, R⁴ and R⁵ are hydrogen, R² is methoxy, R³ is methoxy, n is 1 and R⁶ is a phenyl ring, which is located in the 4-position of the pyridine ring and which carries 1 or 2 radicals R¹¹ as defined in the rows of Table A;

### Table 38

Compounds of the formula I-A.c in which R¹, R⁴ and R⁵ are hydrogen, R² is methyl, R³ is hydrogen, n is 1 and R⁶ is a phenyl ring, which is located in the 4-position of the pyridine ring and which carries 1 or 2 radicals R¹¹ as defined in the rows of Table A;

### Table 39

Compounds of the formula I-A.c in which R¹, R⁴ and R⁵ are hydrogen, R² is hydrogen, R³ is methyl, n is 1 and R⁶ is a phenyl ring, which is located in the 4-position of the pyridine ring and which carries 1 or 2 radicals R¹¹ as defined in the rows of Table A;

### Table 40

Compounds of the formula I-A.c in which R¹, R⁴ and R⁵ are hydrogen, R² is methyl, R³ is methyl, n is 1 and R⁶ is a phenyl ring, which is located in the 4-position of the pyridine ring and which carries 1 or 2 radicals R¹¹ as defined in the rows of Table A;

### Table 41

Compounds of the formula I-A.c in which R¹, R⁴ and R⁵ are hydrogen, R² is hydrogen, R³ is OCHF₂, n is 1 and R⁶ is a phenyl ring, which is located in the 4-position of the pyridine ring and which carries 1 or 2 radicals R¹¹ as defined in the rows of Table A;

### Table 42

Compounds of the formula I-A.c in which R¹, R⁴ and R⁵ are hydrogen, R² is OCHF₂, R³ is hydrogen, n is 1 and R⁶ is a phenyl ring, which is located in the 4-position of the pyridine ring and which carries 1 or 2 radicals R¹¹ as defined in the rows of Table A;

### Table 43

Compounds of the formula I-B.a in which R¹, R², R³, R⁴ and R⁵ are hydrogen, n is 1 and R⁶ is a phenyl ring, which is located in the 6-position of the pyridine ring and which carries 1 or 2 radicals R¹¹ as defined in the rows of Table A;

### Table 44

Compounds of the formula I-B.a in which R¹, R⁴ and R⁵ are hydrogen, R² is hydrogen, R³ is chlorine, n is 1 and R⁶ is a phenyl ring, which is located in the 6-position of the pyridine ring and which carries 1 or 2 radicals R¹¹ as defined in the rows of Table A;

### Table 45

Compounds of the formula I-B.a in which R¹, R⁴ and R⁵ are hydrogen, R² is chlorine, R³ is hydrogen, n is 1 and R⁶ is a phenyl ring, which is located in the 6-position of the pyridine ring and which carries 1 or 2 radicals R¹¹ as defined in the rows of Table A;

### Table 46

Compounds of the formula I-B.a in which R¹, R⁴ and R⁵ are hydrogen, R² is chlorine, R³ is chlorine, n is 1 and R⁶ is a phenyl ring, which is located in the 6-position of the pyridine ring and which carries 1 or 2 radicals R¹¹ as defined in the rows of Table A;

### Table 47

Compounds of the formula I-B.a in which R¹, R⁴ and R⁵ are hydrogen, R² is methoxy, R³ is methyl, n is 1 and R⁶ is a phenyl ring, which is located in the 6-position of the pyridine ring and which carries 1 or 2 radicals R¹¹ as defined in the rows of Table A;

### Table 48

Compounds of the formula I-B.a in which R¹, R⁴ and R⁵ are hydrogen, R² is methyl, R³ is methoxy, n is 1 and R⁶ is a phenyl ring, which is located in the 6-position of the pyridine ring and which carries 1 or 2 radicals R¹¹ as defined in the rows of Table A;

### Table 49

Compounds of the formula I-B.a in which R¹, R⁴ and R⁵ are hydrogen, R² is hydrogen, R³ is methoxy, n is 1 and R⁶ is a phenyl ring, which is located in the 6-position of the pyridine ring and which carries 1 or 2 radicals R¹¹ as defined in the rows of Table A;

### Table 50

Compounds of the formula I-B.a in which R¹, R⁴ and R⁵ are hydrogen, R² is methoxy, R³ is hydrogen, n is 1 and R⁶ is a phenyl ring, which is located in the 6-position of the pyridine ring and which carries 1 or 2 radicals R¹¹ as defined in the rows of Table A;

### Table 51

Compounds of the formula I-B.a in which R¹, R⁴ and R⁵ are hydrogen, R² is methoxy, R³ is methoxy, n is 1 and R⁶ is a phenyl ring, which is located in the 6-position of the pyridine ring and which carries 1 or 2 radicals R¹¹ as defined in the rows of Table A;

### Table 52

Compounds of the formula I-B.a in which R¹, R⁴ and R⁵ are hydrogen, R² is methyl, R³ is hydrogen, n is 1 and R⁶ is a phenyl ring, which is located in the 6-position of the pyridine ring and which carries 1 or 2 radicals R¹¹ as defined in the rows of Table A;

### Table 53

Compounds of the formula I-B.a in which R¹, R⁴ and R⁵ are hydrogen, R² is hydrogen, R³ is methyl, n is 1 and R⁶ is a phenyl ring, which is located in the 6-position of the pyridine ring and which carries 1 or 2 radicals R¹¹ as defined in the rows of Table A;

### Table 54

Compounds of the formula I-B.a in which R¹, R⁴ and R⁵ are hydrogen, R² is methyl, R³ is methyl, n is 1 and R⁶ is a phenyl ring, which is located in the 6-position of the pyridine ring and which carries 1 or 2 radicals R¹¹ as defined in the rows of Table A;

### Table 55

Compounds of the formula I-B.a in which R¹, R⁴ and R⁵ are hydrogen, R² is hydrogen, R³ is OCHF₂, n is 1 and R⁶ is a phenyl ring, which is located in the 6-position of the pyridine ring and which carries 1 or 2 radicals R¹¹ as defined in the rows of Table A;

### Table 56

Compounds of the formula I-B.a in which R¹, R⁴ and R⁵ are hydrogen, R² is OCHF₂, R³ is hydrogen, n is 1 and R⁶ is a phenyl ring, which is located in the 6-position of the pyridine ring and which carries 1 or 2 radicals R¹¹ as defined in the rows of Table A;

### Table 57

Compounds of the formula I-B.b in which R¹, R², R³, R⁴ and R⁵ are hydrogen, n is 1 and R⁶ is a phenyl ring, which is located in the 5-position of the pyridine ring and which carries 1 or 2 radicals R¹¹ as defined in the rows of Table A;

### Table 58

Compounds of the formula I-B.b in which R¹, R⁴ and R⁵ are hydrogen, R² is hydrogen, R³ is chlorine, n is 1 and R⁶ is a phenyl ring, which is located in the 5-position of the pyridine ring and which carries 1 or 2 radicals R¹¹ as defined in the rows of Table A;

### Table 59

Compounds of the formula I-B.b in which R¹, R⁴ and R⁵ are hydrogen, R² is chlorine, R³ is hydrogen, n is 1 and R⁶ is a phenyl ring, which is located in the 5-position of the pyridine ring and which carries 1 or 2 radicals R¹¹ as defined in the rows of Table A;

### Table 60

Compounds of the formula I-B.b in which R¹, R⁴ and R⁵ are hydrogen, R² is chlorine, R³ is chlorine, n is 1 and R⁶ is a phenyl ring, which is located in the 5-position of the pyridine ring and which carries 1 or 2 radicals R¹¹ as defined in the rows of Table A;

### Table 61

Compounds of the formula I-B.b in which R¹, R⁴ and R⁵ are hydrogen, R² is methoxy, R³ is methyl, n is 1 and R⁶ is a phenyl ring, which is located in the 5-position of the pyridine ring and which carries 1 or 2 radicals R¹¹ as defined in the rows of Table A;

### Table 62

Compounds of the formula I-B.b in which R¹, R⁴ and R⁵ are hydrogen, R² is methyl, R³ is methoxy, n is 1 and R⁶ is a phenyl ring, which is located in the 5-position of the pyridine ring and which carries 1 or 2 radicals R¹¹ as defined in the rows of Table A;

### Table 63

Compounds of the formula I-B.b in which R¹, R⁴ and R⁵ are hydrogen, R² is hydrogen, R³ is methoxy, n is 1 and R⁶ is a phenyl ring, which is located in the 5-position of the pyridine ring and which carries 1 or 2 radicals R¹¹ as defined in the rows of Table A;

### Table 64

Compounds of the formula I-B.b in which R¹, R⁴ and R⁵ are hydrogen, R² is methoxy, R³ is hydrogen, n is 1 and R⁶ is a phenyl ring, which is located in the 5-position of the pyridine ring and which carries 1 or 2 radicals R¹¹ as defined in the rows of Table A;

### Table 65

Compounds of the formula I-B.b in which R¹, R⁴ and R⁵ are hydrogen, R² is methoxy, R³ is methoxy, n is 1 and R⁶ is a phenyl ring, which is located in the 5-position of the pyridine ring and which carries 1 or 2 radicals R¹¹ as defined in the rows of Table A;

### Table 66

Compounds of the formula I-B.b in which R¹, R⁴ and R⁵ are hydrogen, R² is methyl, R³ is hydrogen, n is 1 and R⁶ is a phenyl ring, which is located in the 5-position of the pyridine ring and which carries 1 or 2 radicals R¹¹ as defined in the rows of Table A;

### Table 67

Compounds of the formula I-B.b in which R¹, R⁴ and R⁵ are hydrogen, R² is hydrogen, R³ is methyl, n is 1 and R⁶ is a phenyl ring, which is located in the 5-position of the pyridine ring and which carries 1 or 2 radicals R¹¹ as defined in the rows of Table A;

### Table 68

Compounds of the formula I-B.b in which R¹, R⁴ and R⁵ are hydrogen, R² is methyl, R³ is methyl, n is 1 and R⁶ is a phenyl ring, which is located in the 5-position of the pyridine ring and which carries 1 or 2 radicals R¹¹ as defined in the rows of Table A;

### Table 69

Compounds of the formula I-B.b in which R¹, R⁴ and R⁵ are hydrogen, R² is hydrogen, R³ is OCHF₂, n is 1 and R⁶ is a phenyl ring, which is located in the 5-position of the pyridine ring and which carries 1 or 2 radicals R¹¹ as defined in the rows of Table A;

### Table 70

Compounds of the formula I-B.b in which R¹, R⁴ and R⁵ are hydrogen, R² is OCHF₂, R³ is hydrogen, n is 1 and R⁶ is a phenyl ring, which is located in the 5-position of the pyridine ring and which carries 1 or 2 radicals R¹¹ as defined in the rows of Table A

### Table 71

Compounds of the formula I-C.a in which R¹, R², R³, R⁴ and R⁵ are hydrogen n is 1 and R⁶ is a phenyl ring, which is located in the 2-position of the pyridine ring and which carries 1 or 2 radicals R¹¹ as defined in the rows of Table A;

### Table 72

Compounds of the formula I-C.a in which R¹, R⁴ and R⁵ are hydrogen, R² is chlorine, R³ is hydrogen, n is 1 and R⁶ is a phenyl ring, which is located in the 2-position of the pyridine ring and which carries 1 or 2 radicals R¹¹ as defined in the rows of Table A;

### Table 73

Compounds of the formula I-C.a in which R¹, R⁴ and R⁵ are hydrogen, R² is chlorine, R³ is chlorine, n is 1 and R⁶ is a phenyl ring, which is located in the 2-position of the pyridine ring and which carries 1 or 2 radicals R¹¹ as defined in the rows of Table A;

### Table 74

Compounds of the formula I-C.a in which R¹, R⁴ and R⁵ are hydrogen, R² is methoxy, R³ is methyl, n is 1 and R⁶ is a phenyl ring, which is located in the 2-position of the pyridine ring and which carries 1 or 2 radicals R¹¹ as defined in the rows of Table A;

### Table 75

Compounds of the formula I-C.a in which R¹, R⁴ and R⁵ are hydrogen, R² is methyl, R³ is methoxy, n is 1 and R⁶ is a phenyl ring, which is located in the 2-position of the pyridine ring and which carries 1 or 2 radicals R¹¹ as defined in the rows of Table A;

### Table 76

Compounds of the formula I-C.a in which R¹, R⁴ and R⁵ are hydrogen, R² is hydrogen, R³ is methoxy, n is 1 and R⁶ is a phenyl ring, which is located in the 2-position of the pyridine ring and which carries 1 or 2 radicals R¹¹ as defined in the rows of Table A;

### Table 77

Compounds of the formula I-C.a in which R¹, R⁴ and R⁵ are hydrogen, R² is methoxy, R³ is hydrogen, n is 1 and R⁶ is a phenyl ring, which is located in the 2-position of the pyridine ring and which carries 1 or 2 radicals R¹¹ as defined in the rows of Table A;

### Table 78

Compounds of the formula I-C.a in which R¹, R⁴ and R⁵ are hydrogen, R² is methoxy, R³ is methoxy, n is 1 and R⁶ is a phenyl ring, which is located in the 2-position of the pyridine ring and which carries 1 or 2 radicals R¹¹ as defined in the rows of Table A;

### Table 79

Compounds of the formula I-C.a in which R¹, R⁴ and R⁵ are hydrogen, R² is methyl, R³ is hydrogen, n is 1 and R⁶ is a phenyl ring, which is located in the 2-position of the pyridine ring and which carries 1 or 2 radicals R¹¹ as defined in the rows of Table A;

### Table 80

Compounds of the formula I-C.a in which R¹, R⁴ and R⁵ are hydrogen, R² is hydrogen, R³ is methyl, n is 1 and R⁶ is a phenyl ring, which is located in the 2-position of the pyridine ring and which carries 1 or 2 radicals R¹¹ as defined in the rows of Table A;

### Table 81

Compounds of the formula I-C.a in which R¹, R⁴ and R⁵ are hydrogen, R² is methyl, R³ is methyl, n is 1 and R⁶ is a phenyl ring, which is located in the 2-position of the pyridine ring and which carries 1 or 2 radicals R¹¹ as defined in the rows of Table A;

### Table 82

Compounds of the formula I-C.a in which R¹, R⁴ and R⁵ are hydrogen, R² is hydrogen, R³ is OCHF₂, n is 1 and R⁶ is a phenyl ring, which is located in the 2-position of the pyridine ring and which carries 1 or 2 radicals R¹¹ as defined in the rows of Table A;

### Table 83

Compounds of the formula I-C.a in which R¹, R⁴ and R⁵ are hydrogen, R² is OCHF₂, R³ is hydrogen, n is 1 and R⁶ is a phenyl ring, which is located in the 2-position of the pyridine ring and which carries 1 or 2 radicals R¹¹ as defined in the rows of Table A

### Table 84

Compounds of the formula I-C.a in which R¹, R², R³, R⁴ and R⁵ are hydrogen, n is 1 and R⁶ is a phenyl ring, which is located in the 2-position of the pyridine ring and which carries 1 or 2 radicals R¹¹ as defined in the rows of Table A;

### Table 85

Compounds of the formula I-A.a in which R¹, R⁴ and R⁵ are hydrogen, R² and R³ together form a moiety —CH═CH—CH═CH—, n is 1 and R⁶ is a phenyl ring, which is located in the 6-position of the pyridine ring and which carries 1 or 2 radicals R¹¹ as defined in the rows of Table A;

### Table 86

Compounds of the formula I-A.b in which R¹, R⁴ and R⁵ are hydrogen, R² and R³ together form a moiety —CH═CH—CH═CH—, n is 1 and R⁶ is a phenyl ring, which is located in the 5-position of the pyridine ring and which carries 1 or 2 radicals R¹¹ as defined in the rows of Table A;

### Table 87

Compounds of the formula I-A.c in which R¹, R⁴ and R⁵ are hydrogen, R² and R³ together form a moiety —CH═CH—CH═CH—, n is 1 and R⁶ is a phenyl ring, which is located in the 4-position of the pyridine ring and which carries 1 or 2 radicals R¹¹ as defined in the rows of Table A;

### Table 88

Compounds of the formula I-B.a in which R¹, R⁴ and R⁵ are hydrogen, R² and R³ together form a moiety —CH═CH—CH═CH—, n is 1 and R⁶ is a phenyl ring, which is located in the 6-position of the pyridine ring and which carries 1 or 2 radicals R¹¹ as defined in the rows of Table A;

### Table 89

Compounds of the formula I-B.b in which R¹, R⁴ and R⁵ are hydrogen, R² and R³ together form a moiety —CH═CH—CH═CH—, n is 1 and R⁶ is a phenyl ring, which is located in the 5-position of the pyridine ring and which carries 1 or 2 radicals R¹¹ as defined in the rows of Table A;

### Table 90

Compounds of the formula I-C.a in which R¹, R⁴ and R⁵ are hydrogen, R² and R³ together form a moiety —CH═CH—CH═CH—, n is 1 and R⁶ is a phenyl ring, which is located in the 2-position of the pyridine ring and which carries 1 or 2 radicals R¹¹ as defined in the rows of Table A;

TABLE A

| No. | R¹¹ |
|---|---|
| 1 | 2-F |
| 2 | 3-F |
| 3 | 4-F |
| 4 | 2-F, 3-F |
| 5 | 2-F, 4-F |
| 6 | 3-F, 4-F |
| 7 | 2-Cl |
| 8 | 3-Cl |
| 9 | 4-Cl |
| 10 | 2-Cl, 3-Cl |
| 11 | 2-Cl, 4-Cl |
| 12 | 3-Cl, 4-Cl |
| 13 | 2-CH₃ |
| 14 | 3-CH₃ |
| 15 | 4-CH₃ |
| 16 | 2-CH₃, 3-CH₃ |
| 17 | 2-CH₃, 4-CH₃ |
| 18 | 3-CH₃, 4-CH₃ |
| 19 | 2-C₂H₅ |
| 20 | 3-C₂H₅ |
| 21 | 4-C₂H₅ |
| 22 | 2-C₂H₅, 3-C₂H₅ |
| 23 | 2-C₂H₅, 4-C₂H₅ |
| 24 | 3-C₂H₅, 4-C₂H₅ |
| 25 | 2-CH₂CH₂CH₃ |
| 26 | 3-CH₂CH₂CH₃ |
| 27 | 4-CH₂CH₂CH₃ |
| 28 | 2-CH₂CH₂CH_{3,} 3-CH₂CH₂CH₃ |
| 29 | 2-CH₂CH₂CH_{3,} 4-CH₂CH₂CH₃ |
| 30 | 3-CH₂CH₂CH_{3,} 4-CH₂CH₂CH₃ |
| 31 | 2-CH(CH₃)₂ |
| 32 | 3-CH(CH₃)₂ |
| 33 | 4-CH(CH₃)₂ |
| 34 | 2-CH(CH₃)₂, 3-CH(CH₃)₂ |
| 35 | 2-CH(CH₃)₂, 4-CH(CH₃)₂ |
| 36 | 3-CH(CH₃)₂, 4-CH(CH₃)₂ |
| 37 | 4-C(CH₃)₃ |
| 38 | 2-CF₃ |
| 39 | 3-CF₃ |
| 40 | 4-CF₃ |
| 41 | 2-CF₃, 3-CF₃ |
| 42 | 2-CF₃, 4-CF₃ |
| 43 | 3-CF₃, 4-CF₃ |
| 44 | 2-C₂F₅ |
| 45 | 3-C₂F₅ |
| 46 | 4-C₂F₅ |
| 47 | 2-C₂F₅, 3-C₂F₅ |
| 48 | 2-C₂F₅, 4-C₂F₅ |
| 49 | 3-C₂F₅, 4-C₂F₅ |
| 50 | 2-OH |
| 51 | 3-OH |
| 52 | 4-OH |
| 53 | 2-OH, 3-OH |
| 54 | 2-OH, 4-OH |
| 55 | 3-OH, 4-OH |
| 56 | 2-OCH₃ |
| 57 | 3-OCH₃ |
| 58 | 4-OCH₃ |
| 59 | 2-OCH₃, 3-OCH₃ |
| 60 | 2-OCH₃, 4-OCH₃ |
| 61 | 3-OCH₃, 4-OCH₃ |
| 62 | 2-OCF₃ |
| 63 | 3-OCF₃ |
| 64 | 4-OCF₃ |
| 65 | 2-OCF₃, 3-OCF₃ |
| 66 | 2-OCF₃, 4-OCF₃ |
| 67 | 3-OCF₃, 4-OCF₃ |
| 68 | 2-OC₂F₅ |
| 69 | 3-OC₂F₅ |
| 70 | 4-OC₂F₅ |
| 71 | 2-OC₂F₅, 3-OC₂F₅ |
| 72 | 2-OC₂F₅, 4-OC₂F₅ |
| 73 | 3-OC₂F₅, 4-OC₂F₅ |
| 74 | 2-NO₂ |
| 75 | 3-NO₂ |
| 76 | 4-NO₂ |
| 77 | 2-NO₂, 3-NO₂ |
| 78 | 2-NO₂, 4-NO₂ |
| 79 | 3-NO₂, 4-NO₂ |
| 80 | 2-CN |
| 81 | 3-CN |
| 82 | 4-CN |
| 83 | 2-CN, 3-CN |
| 84 | 2-CN, 4-CN |
| 85 | 3-CN, 4-CN |
| 86 | 2-(CO—OCH₃) |
| 87 | 3-(CO—OCH₃) |
| 88 | 4-(CO—OCH₃) |
| 89 | 2-(CO—OC₂H₅) |
| 90 | 3-(CO—OC₂H₅) |
| 91 | 4-(CO—OC₂H₅) |
| 92 | 2-CHO |
| 93 | 3-CHO |
| 94 | 4-CHO |
| 95 | 2-(CO—CH₃) |
| 96 | 3-(CO—CH₃) |
| 97 | 4-(CO—CH₃) |
| 98 | 2-(CO—NH₂) |
| 99 | 3-(CO—NH₂) |
| 100 | 4-(CO—NH₂) |
| 101 | 2-[C(CH₃)═N—OCH₃] |
| 102 | 3-[C(CH₃)═N—OCH₃] |
| 103 | 4-[C(CH₃)═N—OCH₃] |
| 104 | 2-[C(CH₃)═N—OC₂H₅] |
| 105 | 3-[C(CH₃)═N—OC₂H₅] |
| 106 | 4-[C(CH₃)═N—OC₂H₅] |
| 107 | 2-SCH₃ |
| 108 | 3-SCH₃ |
| 109 | 4-SCH₃ |
| 110 | 2-(SO₂—CH₃) |
| 111 | 3-(SO₂—CH₃) |
| 112 | 4-(SO₂—CH₃) |
| 113 | 2-(SO—CH₃) |
| 114 | 3-(SO—CH₃) |
| 115 | 4-(SO—CH₃) |
| 116 | 2-[N(CH₃)₂] |
| 117 | 3-[N(CH₃)₂] |
| 118 | 4-[N(CH₃)₂] |
| 119 | 2-[Si(CH₃)₃] |
| 120 | 3-[Si(CH₃)₃] |
| 121 | 4-[Si(CH₃)₃] |
| 122 | 2-F, 3-Cl |
| 123 | 2-F, 4-Cl |
| 124 | 2-F, 5-Cl |
| 125 | 2-F, 6-Cl |
| 126 | 3-F, 2-Cl |
| 127 | 3-F, 4-Cl |
| 128 | 3-F, 5-Cl |
| 129 | 4-F, 2-Cl |
| 130 | 4-F, 3-Cl |
| 131 | 4-F, 2-CH₃ |
| 132 | 4-Cl, 2-CH₃ |
| 133 | 2-Cl, 4-OCH₃ |
| 134 | 3-Cl, 4-OCH₃ |
| 135 | 2-F, 4-OCH₃ |
| 136 | 3-F, 4-OCH₃ |
| 137 | 3,4 (O—CH₂—O) |

The compounds I according to the invention can be prepared by analogy to the methods described in the art.

Advantageously, they are obtained from pyridine derivatives of the formula II. A suitable process for the preparation of the compounds I comprises the reaction of compounds II with sulfonic acids or sulfonic acid derivatives of the formula III, under basic conditions as described in the following reaction scheme:

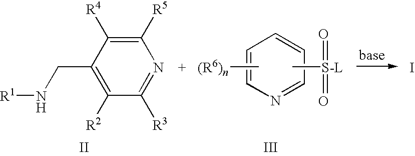

In formulae II and III, n and the radicals R¹, R², R³, R⁴, R⁵, and R⁶ are as defined above. In formula III, L is a suitable leaving group such as hydroxyl or halogen, preferably chlorine.

This reaction is usually carried out at temperatures of from (−30)° C. to 120° C., preferably from (−10)° C. to 100° C., in an inert organic solvent in the presence of a base [cf. Lieb. Ann. Chem. 641 (1990)].

Suitable solvents include aliphatic hydrocarbons, such as pentane, hexane, cyclohexane and petroleum ether, aromatic hydrocarbons, such as toluene, o-, m- and p-xylene, halogenated hydrocarbons, such as methylene chloride, chloroform and chlorobenzene, ethers, such as diethyl ether, diisopropyl ether, tert.-butyl methyl ether, dioxane, anisole and tetrahydrofuran, nitriles, such as acetonitrile and propionitrile, ketones, such as acetone, methyl ethyl ketone, diethyl ketone and tert.-butyl methyl ketone, and also dimethyl sulfoxide, dimethylformamide and dimethylacetamide, particularly preferably diisopropyl ether, diethyl ether and tetrahydrofuran. It is also possible to use mixtures of the solvents mentioned.

Suitable bases are, in general, inorganic compounds, such as alkali metal and alkaline earth metal hydroxides, such as lithium hydroxide, sodium hydroxide, potassium hydroxide and calcium hydroxide, alkali metal and alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal and alkaline earth metal carbonates, such as lithium carbonate, potassium carbonate and calcium carbonate, and also alkali metal bicarbonates, such as sodium bicarbonate, moreover organic bases, for example tertiary amines, such as trimethylamine, triethylamine, triisopropylethylamine and N-methylpiperidine, pyridine, substituted pyridines, such as collidine, lutidine and 4-dimethylaminopyridine, and also bicyclic amines. Particular preference is given to pyridine, triethylamine and potassium carbonate.

The bases are generally employed in catalytic amounts; however, they can preferably be employed in equimolar amounts, in particular in excess or, if appropriate, as solvent.

The starting materials are generally reacted with one another in equimolar amounts. In terms of yield, it may be advantageous to use an excess of II, based on III.

Compounds, wherein R⁶ is optionally substituted phenyl or hetaryl may also be prepared from compounds I, wherein R⁶ is halogen, in particular bromine by a coupling reaction such as a Stille-coupling or under conditions of a Suzuki-Coupling, e.g. by the reaction shown in the following reaction scheme:

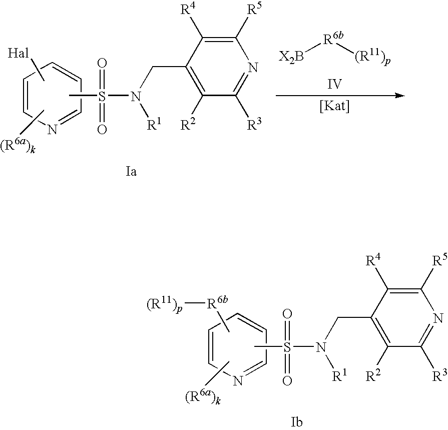

In the formulae Ia, Ib and IV, the variables R¹, R², R³, R⁴, R⁵, and R¹¹ are as defined above. The variable k is 0 or 1. The variable p is 0, 1, 2, or 3. R^{6a} has one of the meanings given for R⁶, except for phenyl or 5- or 6-membered hetaryl. R^{6b} is phenyl or 5- or 6-membered hetaryl. Hal in formula Ia is halogen, in particular bromine. X in Formula IV is OH or C₁-C₄-alkoxy. Kat is a transition metal catalyst, in particular a Pd-catalyst. Reaction conditions can be taken from the working examples or from Suzuki et al., Chem. Rev, 1995, 95, 2457-2483 and the literature cited therein.

The intermediate III can be prepared from the respective pyridylhalide V by treatment with alkylmagnesiumhalogenide such as iPrMgCl, SO₂ and SO₂Cl₂ as shown in the scheme below.

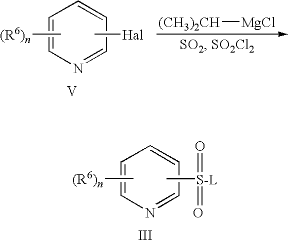

The starting materials required for preparing the compounds I are commercially available or known in the art or they can be prepared by analogy to the methods described in the art.

For example, aminomethylpyridine compounds of the formula II in which one or more of the radicals R², R³, R⁴ or R⁵ is/are different from hydrogen and, such as (halo)alkoxy, (halo)alkylthio, (halo)alkyl, alkenyl, trialkylsilyl or alkynyl may be prepared starting from halopyridinecarbonitriles by replacing a halogen radical against a radical different from halogen, by conventional nucleophilic substitution reaction or by a coupling reaction, e.g. by treatment with suitable nucleophile such as HNR¹⁷R¹⁸, (halo)alkoxide, (halo)alkylthio, a metal organic compound, optionally in the presence of a transition metal catalyst, to obtain the corresponding substituted carbonitrile [cf. Journal of Medicinal Chemistry, 22(11), 1284-90; 1979; U.S. Pat. No. 4,558,134, Synthesis, (6), 763-768; 1996 and Heterocycles, 41(4), 675-88; 1995], and subsequent hydrogenation of the C≡N radical to obtain the corresponding aminomethylpyridine compound II, wherein R¹ is hydrogen [cf. Heterocycles, 41(4), 675-88; 1995; Recueil des Travaux Chimiques des Pays-Bas et de la Belgique, 52, 55-60; 1933; Acta Poloniae Pharmaceutica, 32(3), 265-8; 1975; Journal of Medicinal Chemistry, 24(1), 115-17; 1981, P 49173, Heterocycles, 41(4), 675-88; 1995, Angewandte Chemie, International Edition, 43(37), 4902-4906; 2004; Journal of Heterocyclic Chemistry, 19(6), 1551-2; 1982]. The subsequent alkylation of the amino methyl nitrogen yields compounds, wherein R¹ is different from hydrogen.

The reaction mixtures are worked up in the customary manner, for example by mixing with water, separating the phases and, if appropriate, chromatographic purification of the crude products. Some of the intermediates and end products are obtained in the form of colorless or slightly brownish viscous oils, which can be purified or freed from volatile components under reduced pressure and at moderately elevated temperature. If the intermediates and end products are obtained as solids, purification can also be carried out by recrystallization or digestion.

The N-oxides may be prepared from the compounds I according to conventional oxidation methods, for example by treating pyridine compounds I with an organic peracid such as metachloroperbenzoic acid [Journal of Medicinal Chemistry, 38(11), 1892-903; 1995, WO 03/64572]; or with inorganic oxidizing agents such as hydrogen peroxide [cf. Journal of Heterocyclic Chemistry, 18(7), 1305-8; 1981] or oxone [cf. Journal of the American Chemical Society, 123(25), 5962-5973; 2001]. The oxidation may lead to pure mono-, bis- or tris-N-oxides or to a mixture of different N-oxides, which can be separated by conventional methods such as chromatography. Preferably one or two of the pyridine nitrogens in compounds I are oxidized to the corresponding mono- or bis-N-oxides.

If individual compounds I cannot be obtained by the routes described above, they can be prepared by derivatization of other compounds I.

If the synthesis yields mixtures of isomers, a separation is generally not necessarily required since in some cases the individual isomers can be interconverted during work-up for use or during application (for example under the action of light, acids or bases). Such conversions may also take place after use, for example in the treatment of plants in the treated plant, or in the harmful fungus or pest to be controlled.

The compounds I are suitable as fungicides. They are distinguished by an outstanding effectiveness against a broad spectrum of phytopathogenic fungi, especially from the classes of the Ascomycetes, Deuteromycetes, Oomycetes and Basidiomycetes. Some are systemically effective and they can be used in crop protection as foliar fungicides, fungicides for seed dressing and soil fungicides.

They are particularly important in the control of a multitude of fungi on various cultivated plants, such as wheat, rye, barley, oats, rice, corn, grass, bananas, cotton, soybeans, coffee, sugar cane, grapevines, fruit and ornamental plants, and vegetables, such as cucumbers, beans, tomatoes, potatoes and cucurbits, and on the seeds of these plants.

They are especially suitable for controlling the following plant diseases:
Alternaria species on fruit, rape, sugar beets, rice and vegetables (e.g. A. solani or A. alternata on potatoes and tomatoes),
Aphanomyces species on sugar beets and vegetables,
Ascochyta species on cereals and vegetables,
Bipolaris and Drechslera species on cereals, corn, rice and lawns (e.g. D. maydis on corn),
Blumeria graminis (powdery mildew) on cereals,
Botrytis cinerea (gray mold) on strawberries, vegetables, ornamental plants and grapevines,
Bremia lactucae on lettuce,
Cerospora species on corn, soybeans, rice and sugar beets,
Cochliobolus species on corn, cereals, rice (e.g. Cochliobolus sativus on cereals, Cochliobolus miyabeanus on rice),
Colletotricum species on soybeans and cotton,
Drechslera species, Pyrenophora species on corn, cereals, rice and lawn (e.g. D. teres on barley or D. tritici-repentis on wheat).
Esca on grapevines, caused by Phaeoacremonium chlamydosporium, Ph. Aleophilum and Formitipora punctata (syn. Phellinus punctatus),
Elsinoe ampelina on grapevines
Exserohilum species on corn,
Erysiphe cichoracearum and Sphaerotheca fuliginea on cucurbits,
Erysiphe (syn. Uncinula) necator on grapevine,
Fusarium and Verticillium species on various plants (e.g. F. graminearum or F. culmorum on cereals or F. oxysporum on various plants, e.g. tomatoes),
Gaeumanomyces graminis on cereals,
Gibberella species on cereals and rice (e.g. Gibberella fujikuroi on rice),
Glomerella cingulata on grapevines and other plants,
Grainstaining complex on rice,
Guignardia budwelli on grapevines,
Helminthosporium species on corn and rice,
Isariopsis clavispora on grapevines,
Michrodochium nivale on cereals,
Mycosphaerella species on cereals, bananas and peanuts (e.g. M. graminicola on wheat or M. fijiesis on bananas),
Peronospora species on cabbage and onion plants (e.g. P. brassicae on cabbage or P. destructor on onions),
Phakopsara pachyrhizi and Phakopsara meibomiae on soybeans,
Phomopsis species on soybeans and sun flowers,
Phytophthora infestans on potatoes and tomatoes,
Phytophthora species on various plants (e.g. P. capsici on paprika),
Plasmopara viticola on grapevines,
Podosphaera leucotricha on apples,
Pseudocercosporella herpotrichoides on cereals, especially wheat and barley,
Pseudoperonospora on various plants (P. cubensis on cucumber or P. humili on hops),
Pseudopezicula tracheiphilai on grapevines,
Puccinia on various plants (e.g. P. triticina, P. striformins, P. hordei or P. graminis on cereals or P. asparagi on asparagus),
Pyrenophora species on cereals,
Pyricularia oryzae, Corticium sasakii, Sarocladium oryzae, S. attenuatum, Entyloma oryzae on rice,
Pyricularia grisea on lawns and cereals,
Pythium spp. on lawns, rice, corn, cotton, rape, sun flowers, sugar beets, vegetables and other plants (e.g. P. ultiumum on various plants, P. aphanidermatum on lawns),
Rhizoctonia species on cotton, rice, lawns, potatoes, corn, rape, sugar beets, vegetables and on various plants plants (e.g. R. solani on beets and various plants),
Sclerotinia species on rape and sun flowers,
Septoria tritici and Stagonospora nodorum on wheat,
Setospaeria species on corn and lawns,
Sphacelotheca reilinia on corn,
Thievaliopsis species on soybeans and cotton,
Tilletia species on cereals,
Uncinula necator on grapevines,
Ustilago species on cereals, corn and sugar cane (e.g. U. maydis on corn), and
Venturia species (scab) on apples and pears.

The compounds I are also suitable for controlling harmful fungi in the protection of materials (e.g. wood, paper, paint dispersions, fiber or fabrics) and in the protection of stored products. As to the protection of wood, the following harmful fungi are worthy of note: Ascomycetes such as Ophiostoma spp., Ceratocystis spp., Aureobasidium pullulans, Sclerophoma spp., Chaetomium spp., Humicola spp., Petriella spp., Trichurus spp.; Basidiomycetes such as Coniophora spp., Coriolus spp., Gloeophyllum spp., Lentinus spp., Pleurotus spp., Poria spp., Serpula spp. and Tyromyces spp., Deuteromycetes such as Aspergillus spp., Cladosporium spp., Penicillium spp., Trichoderma spp., Alternaria spp., Paecilomyces spp. and Zygomycetes such as Mucor spp., and in addition in the protection of stored products the following yeast fungi are worthy of note: Candida spp. and Saccharomyces cerevisae.

In addition the compounds of the formula I may also be used in cultures which can tolerate insecticidal or fungal attack due to cultivation, including of genetic engineering.

Furthermore, the compounds of the formula I, their N-oxides and salts, according to the invention show high activity against harmful arthropods. They can be used as pesticides in crop protection and in the sectors of hygiene and the protection of stored products and the veterinary sector.

They may act by contact or may be stomach-acting, or have systemic or residual action. Contact action means that the pest is killed by coming into contact with a compound I or with material that releases compound I. Stomach-acting means that the pest is killed if it ingests a pesticidally effective amount of the compound I or material containing a pesticidally effective amount of compound I. Systemic action means that the compound is absorbed into the plant tissues of treated plant and the pest is controlled, if it eats plant tissue or sucks plant-sap. Compounds I are in particular suitable for controlling insect pests, such as
from the order of Lepidoptera, for example Agrotis ypsilon, Agrotis segetum, Alabama argillacea, Anticarsia gemmatalis, Argyresthia conjugella, Autographa gamma, Bupalus piniarius, Cacoecia murinana, Capua reticulana, Chematobia brumata, Choristoneura fumiferana, Choristoneura occidentalis, Cirphis unipuncta, Cydia pomonella, Dendrolimus pini, Diaphania nitidalis, Diatraea grandiosella, Earias insulana, Elasmopalpus lignosellus, Eupoecilia ambiguella, Evetria bouliana, Feltia subterranea, Galleria mellonella, Grapholitha funebrana, Grapholitha molesta, Heliothis armigera, Heliothis virescens, Heliothis zea, Hellula undalis, Hibernia defoliaria, Hyphantria cunea, Hyponomeuta malinellus, Keiferia lycopersicella, Lambdina fiscellaria, Laphygma exigua, Leucoptera coffeella, Leucoptera scitella, Lithocolletis blancardella, Lobesia botrana, Loxostege sticticalis, Lymantria dispar, Lymantria monacha, Lyonetia clerkella, Malacosoma neustria, Mamestra brassicae, Orgyia pseudotsugata, Ostrinia nubilalis, Panolis flammea, Pectinophora gossypiella, Peridroma saucia, Phalera bucephala, Phthorimaea operculella, Phyllocnistis citrella, Pieris brassicae, Plathypena scabra, Plutella xylostella, Pseudoplusia includens, Rhyacionia frustrana, Scrobipalpula absoluta, Sitotroga cerealella, Sparganothis pilleriana, Spodoptera eridania, Spodoptera frugiperda, Spodoptera littoralis, Spodoptera litura, Thaumatopoea pityocampa, Tortrix viridana, Trichoplusia ni and Zeiraphera canadensis,
from the order of Coleoptera (beetles), for example Agrilus sinuatus, Agriotes lineatus, Agriotes obscurus, Amphimallus solstitialis, Anisandrus dispar, Anthonomus grandis, Anthonomus pomorum, Atomaria linearis, Blastophagus piniperda, Blitophaga undata, Bruchus rufimanus, Bruchus pisorum, Bruchus lentis, Byctiscus betulae, Cassida nebulosa, Cerotoma trifurcata, Ceuthorrhynchus assimilis, Ceuthorrhynchus napi, Chaetocnema tibialis, Conoderus vespertinus, Crioceris asparagi, Diabrotica longicornis, Diabrotica 12-punctata, Diabrotica virgifera, Epilachna varivestis, Epitrix hirtipennis, Eutinobothrus brasiliensis, Hylobius abietis, Hypera brunneipennis, Hypera postica, Ips typographus, Lema bilineata, Lema melanopus, Leptinotarsa decemlineata, Limonius californicus, Lissorhoptrus oryzophilus, Melanotus communis, Meligethes aeneus, Melolontha hippocastani, Melolontha melolontha, Oulema oryzae, Ortiorrhynchus sulcatus, Otiorrhynchus ovatus, Phaedon cochleariae, Phyllotreta chrysocephala, Phyllophaga sp., Phyllopertha horticola, Phyllotreta nemorum, Phyllotreta striolata, Popillia japonica, Sitona lineatus and Sitophilus granaria,
from the order of Diptera, for example Aedes aegypti, Aedes vexans, Anastrepha ludens, Anopheles maculipennis, Ceratitis capitata, Chrysomya bezziana, Chrysomya hominivorax, Chrysomya macellaria, Contarinia sorghicola, Cordylobia anthropophaga, Culex pipiens, Dacus cucurbitae, Dacus oleae, Dasineura brassicae, Fannia canicularis, Gasterophilus intestinalis, Glossina morsitans, Haematobia irritans, Haplodiplosis equestris, Hylemyia platura, Hypoderma lineata, Liriomyza sativae, Liriomyza trifolii, Lucilia caprina, Lucilia cuprina, Lucilia sericata, Lycoria pectoralis, Mayetiola destructor, Musca domestica, Muscina stabulans, Oestrus ovis, Oscinella frit, Pegomya hysocyami, Phorbia antiqua, Phorbia brassicae, Phorbia coarctata, Rhagoletis cerasi, Rhagoletis pomonella, Tabanus bovinus, Tipula oleracea and Tipula paludosa,
from the order of Thysanoptera (thrips), e.g. Dichromothrips spp., Frankliniella fusca, Frankliniella occidentalis, Frankliniella tritici, Scirtothrips citri, Thrips oryzae, Thrips palmi and Thrips tabaci,
from the order of Hymenoptera e.g. Athalia rosae, Atta cephalotes, Atta sexdens, Atta texana, Hoplocampa minuta, Hoplocampa testudinea, Monomorium pharaonis, Solenopsis geminata and Solenopsis invicta,
from the order of Heteroptera, e.g. Acrosternum hilare, Blissus leucopterus, Cyrtopeltis notatus, Dysdercus cingulatus, Dysdercus intermedius, Eurygaster integriceps, Euschistus impictiventris, Leptoglossus phyllopus, Lygus lineolaris, Lygus pratensis, Nezara viridula, Piesma quadrata, Solubea insularis and Thyanta perditor,
from the order of Homoptera, e.g. Acyrthosiphon onobrychis, Adelges laricis, Aphidula nasturtii, Aphis craccivora, Aphis fabae, Aphis forbesi, Aphis pomi, Aphis gossypii, Aphis grossulariae, Aphis schneideri, Aphis spiraecola, Aphis sambuci, Acyrthosiphon pisum, Aulacorthum solani, Bemisa tabaci, Bemisa argentifolii, Brachycaudus cardui, Brachycaudus helichrysi, Brachycaudus persicae, Brachycaudus prunicola, Brevicoryne brassicae, Capitophorus horni, Cerosipha gossypii, Chaetosiphon fragaefolii, Cryptomyzus ribis, Dreyfusia nordmannianae, Dreyfusia piceae, Dysaphis radicola, Dysaulacorthum pseudosolani, Dysaphis plantaginea, Dysaphis pyri, Empoasca fabae, Hyalopterus pruni, Hyperomyzus lactucae, Macrosiphum avenae, Macrosiphum euphorbiae, Macrosiphon rosae, Megoura viciae, Melanaphis pyrarius, Metopolophium dirhodum, Myzodes persicae, Myzus ascalonicus, Myzus cerasi, Myzus varians, Nasonovia ribis-nigri, Nilaparvata lugens, Pemphigus bursarius, Perkinsiella saccharicida, Phorodon humuli, Psylla mali, Psylla piri, Rhopalomyzus ascalonicus, Rhopalosiphum maidis, Rhopalosiphum padi, Rhopalosiphum insertum, Sappaphis mala, Sappaphis mali, Schizaphis graminum, Schizoneura lanuginosa, Sitobion avenae, Trialeurodes vaporariorum, Toxoptera aurantiiand, and Viteus vitifolii,
from the order of Isoptera (termites), e.g. Calotermes flavicollis, Leucotermes flavipes, Reticulitermes lucifugus und Termes natalensis, and
from the order of Orthoptera, e.g. Acheta domestica, Blatta orientalis, Blattella germanica, Forficula auricularia, Gryllotalpa gryllotalpa, Locusta migratoria, Melanoplus bivittatus, Melanoplus femur-rubrum, Melanoplus mexicanus, Melanoplus sanguinipes, Melanoplus spretus, Nomadacris septemfasciata, Periplaneta americana, Schistocerca americana, Schistocerca peregrina, Stauronotus maroccanus and Tachycines asynamorus.

The compounds of the formula I, their N-oxides and their salts are also useful for controlling arachnids (Arachnoidea), such as acarians (Acarina), e.g. of the families Argasidae, Ixodidae and Sarcoptidae, such as Amblyomma americanum, Amblyomma variegatum, Argas persicus, Boophilus annulatus, Boophilus decoloratus, Boophilus microplus, Dermacentor silvarum, Hyalomma truncatum, Ixodes ricinus, Ixodes rubicundus, Ornithodorus moubata, Otobius megnini, Dermanyssus gallinae, Psoroptes ovis, Rhipicephalus appendiculatus, Rhipicephalus evertsi, Sarcoptes scabiei, and Eriophyidae spp. such as Aculus schlechtendali, Phyllocoptrata oleivora and Eriophyes sheldoni; Tarsonemidae spp. such as Phytonemus pallidus and Polyphagotarsonemus latus; Tenuipalpidae spp. such as Brevipalpus phoenicis; Tetranychidae spp. such as Tetranychus cinnabarinus, Tetranychus kanzawai, Tetranychus pacificus, Tetranychus telarius and Tetranychus urticae, Panonychus ulmi, Panonychus citri, and Oligonychus pratensis.

The compounds of the formula I, their N-oxides and their salts are also useful for controlling nematodes, for example, root gall nematodes, e.g. Meloidogyne hapla, Meloidogyne incognita, Meloidogyne javanica, cyst-forming nematodes, e.g. Globodera rostochiensis, Heterodera avenae, Heterodera glycines, Heterodera schachtii, Heterodera trifolii, stem and leaf nematodes, e.g. Belonolaimus longicaudatus, Ditylenchus destructor, Ditylenchus dipsaci, Heliocotylenchus multicinctus, Longidorus elongatus, Radopholus similis, Rotylenchus robustus, Trichodorus primitivus, Tylenchorhynchus claytoni, Tylenchorhynchus dubius, Pratylenchus neglectus, Pratylenchus penetrans, Pratylenchus curvitatus and Pratylenchus goodeyi.

Compounds of the formula I are particularly useful for controlling insects of the order Lepidoptera.

The compounds I, their N-oxides and salts can be converted into customary formulations (agricultural formulations), e.g. solutions, emulsions, suspensions, dusts, powders, pastes and granules. Therefore the invention also relates to agricultural compositions which comprise a solid or liquid carrier and at least one pyridin-4-ylmethyl-amid compound of the formula I or an N-oxide or an agriculturally acceptable salt thereof. The agricultural compositions of the invention generally comprise between 0.1 and 95%, preferably between 0.5 and 90%, by weight of active compound.

The formulations are prepared in a known manner, e.g. by extending the active ingredient with solvents and/or carriers, if desired using emulsifiers and dispersants. Solvents/auxiliaries, which are suitable, are essentially:
water, aromatic solvents (for example Solvesso products, xylene), paraffins (for example mineral fractions), alcohols (for example methanol, butanol, pentanol, benzyl alcohol), ketones (for example cyclohexanone, gamma-butyrolactone), pyrrolidones (methylpyrrolidone, (NMP), N-octylpyrrolidone (NOP)), acetates (glycol diacetate), glycols, fatty acid dimethylamides, fatty acids and fatty acid esters. In principle, solvent mixtures may also be used.
carriers such as ground natural minerals (e.g. kaolins, clays, talc, chalk) and ground synthetic minerals (e.g. highly disperse silica, silicates); emulsifiers such as nonionic and anionic emulsifiers (e.g. polyoxyethylene fatty alcohol ethers, alkylsulfonates and arylsulfonates) and dispersants such as lignin-sulfite waste liquors and methylcellulose.

Suitable surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenyl ether, ethoxylated isooctylphenol, octylphenol, nonylphenol, alkylphenyl polyglycol ethers, tributylphenyl polyglycol ether, tristearylphenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters, lignin-sulfite waste liquors and methylcellulose.

Substances which are suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dusts can be prepared by mixing or concomitantly grinding the active substances with a solid carrier.

Granules, for example coated granules, impregnated granules and homogeneous granules, can be prepared by binding the active ingredients to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

The active ingredients are employed in a purity of from 90% to 100%, preferably from 95% to 100% (according to NMR spectrum).

The following are examples of formulations:

1. Products for Dilution with Water

### A Water-Soluble Concentrates (SL)

10 parts by weight of a compound according to the invention are dissolved in water or in a water-soluble solvent. As an alternative, wetters or other auxiliaries are added. The active compound dissolves upon dilution with water.

### B Dispersible Concentrates (DC)

20 parts by weight of a compound according to the invention are dissolved in cyclohexanone with addition of a dispersant, for example polyvinylpyrrolidone. Dilution with water gives a dispersion.

### C Emulsifiable Concentrates (EC)

15 parts by weight of a compound according to the invention are dissolved in xylene with addition of calcium dodecylbenzenesulfonate and castor oil ethoxylate (in each case 5%). Dilution with water gives an emulsion.

### D Emulsions (EW, EO)

40 parts by weight of a compound according to the invention are dissolved in xylene with addition of calcium dodecylbenzenesulfonate and castor oil ethoxylate (in each case 5%). This mixture is introduced into water by means of an emulsifying machine (Ultraturrax) and made into a homogeneous emulsion. Dilution with water gives an emulsion.

### E Suspensions (SC, OD)

In an agitated ball mill, 20 parts by weight of a compound according to the invention are comminuted with addition of dispersants, wetters and water or an organic solvent to give a fine active compound suspension. Dilution with water gives a stable suspension of the active compound.

### F Water-Dispersible Granules and Water-Soluble Granules (WG, SG)

50 parts by weight of a compound according to the invention are ground finely with addition of dispersants and wetters and made into water-dispersible or water-soluble granules by means of technical appliances (for example extrusion, spray tower, fluidized bed). Dilution with water gives a stable dispersion or solution of the active compound.

### G Water-Dispersible Powders and Water-Soluble Powders (WP, SP)

75 parts by weight of a compound according to the invention are ground in a rotor-stator mill with addition of dispersants, wetters and silica gel. Dilution with water gives a stable dispersion or solution of the active compound.

### 2. Products to be Applied Undiluted

### H Dustable Powders (DP)

5 parts by weight of a compound according to the invention are ground finely and mixed intimately with 95% of finely divided kaolin. This gives a dustable product.

### I Granules (GR, FG, GG, MG)

0.5 part by weight of a compound according to the invention is ground finely and associated with 95.5% carriers. Current methods are extrusion, spray-drying or the fluidized bed. This gives granules to be applied undiluted.

### J ULV Solutions (UL)

10 parts by weight of a compound according to the invention are dissolved in an organic solvent, for example xylene. This gives a product to be applied undiluted.

The active ingredients can be used as such, in the form of their formulations or the use forms prepared therefrom, e.g. in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; it is intended to ensure in each case the finest possible distribution of the active ingredients according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active ingredient concentrations in the ready-to-use products can be varied within relatively wide ranges. In general, they are from 0.0001 to 10%, preferably from 0.001 to 1%.

The active ingredients may also be used successfully in the ultra-low-volume process (ULV), it being possible to apply formulations comprising over 95% by weight of active ingredient, or even to apply the active ingredient without additives.

The compositions according to the invention can, in the use form as fungicides, also be present together with other active compounds, e.g. with herbicides, insecticides, growth regulators, fungicides or else with fertilizers. Mixing the compounds I or the compositions comprising them in the use form as fungicides with other fungicides results in many cases in an expansion of the fungicidal spectrum of activity being obtained.

The following list of fungicides, in conjunction with which the compounds according to the invention can be used, is intended to illustrate the possible combinations but does not limit them:
acylalanines, such as benalaxyl, metalaxyl, ofurace or oxadixyl,
amine derivatives, such as aldimorph, dodine, dodemorph, fenpropimorph, fenpropidin, guazatine, iminoctadine, spiroxamine or tridemorph,
anilinopyrimidines, such as pyrimethanil, mepanipyrim or cyprodinil,
antibiotics, such as cycloheximide, griseofulvin, kasugamycin, natamycin, polyoxin or streptomycin,
azoles, such as bitertanol, bromoconazole, cyproconazole, difenoconazole, dinitroconazole, enilconazole, epoxiconazole, fenbuconazole, fluquinconazole, flusilazole, flutriafol, hexaconazole, imazalil, ipconazole, metconazole, myclobutanil, penconazole, propiconazole, prochloraz, prothioconazole, simeconazole, tebuconazole, tetraconazole, triadimefon, triadimenol, triflumizole or triticonazole,
dicarboximides, such as iprodione, myclozolin, procymidone or vinclozolin,
dithiocarbamates, such as ferbam, nabam, maneb, mancozeb, metam, metiram, propineb, polycarbamate, thiram, ziram or zineb,
heterocyclic compounds, such as anilazine, benomyl, boscalid, carbendazim, carboxin, oxycarboxin, cyazofamid, dazomet, dithianon, famoxadone, fenamidone, fenarimol, fuberidazole, flutolanil, furametpyr, isoprothiolane, mepronil, nuarimol, picobenzamide, probenazole, proquinazid, pyrifenox, pyroquilon, quinoxyfen, silthiofam, thiabendazole, thifluzamide, thiophanate-methyl, tiadinil, tricyclazole or triforine,
copper fungicides, such as Bordeaux mixture, copper acetate, copper oxychloride or basic copper sulfate,
nitrophenyl derivatives, such as binapacryl, dinocap, dinobuton or nitrophthal-isopropyl,
phenylpyrroles, such as fenpiclonil or fludioxonil,
sulfur,
other fungicides, such as acibenzolar-S-methyl, benthiavalicarb, carpropamid, chlorothalonil, cyflufenamid, cymoxanil, diclomezine, diclocymet, diethofencarb, edifenphos, ethaboxam, fenhexamid, fentin acetate, fenoxanil, ferimzone, fluazinam, fosetyl, fosetyl-aluminum, phosphorous acid, iprovalicarb, hexachlorobenzene, metrafenone, pencycuron, penthropyrad, propamocarb, phthalide, toloclofos-methyl, quintozene or zoxamide,
strobilurins, such as azoxystrobin, dimoxystrobin, enestroburin, fluoxastrobin, kresoxim-methyl, metominostrobin, orysastrobin, picoxystrobin, pyraclostrobin or trifloxystrobin,
sulfenic acid derivatives, such as captafol, captan, dichlofluanid, folpet or tolylfluanid,
cinnamides and analogous compounds, such as dimethomorph, flumetover or flumorph.

Compositions of this invention may also contain other active ingredients, for example other pesticides such as insecticides and herbicides, fertilizers such as ammonium nitrate, urea, potash, and superphosphate, phytotoxicants and plant growth regulators, safeners and nematicides. These additional ingredients may be used sequentially or in combination with the above-described compositions, if appropriate also added only immediately prior to use (tank mix). For example, the plant(s) may be sprayed with a composition of this invention either before or after being treated with other active ingredients.

These agents usually are admixed with the agents according to the invention in a weight ratio of 1:100 to 100:1.

The following list of pesticides together with which the compounds according to the invention can be used, is intended to illustrate the possible combinations, but not to impose any limitation:

A.1. Organo(thio)phosphates: e.g. acephate, azamethiphos, azinphos-methyl, chlorpyrifos, chlorpyrifos-methyl, chlorfenvinphos, diazinon, dichlorvos, dicrotophos, dimethoate, disulfoton, ethion, fenitrothion, fenthion, isoxathion, malathion, methamidophos, methidathion, methyl-parathion, mevinphos, monocrotophos, oxydemeton-methyl, paraoxon, parathion, phenthoate, phosalone, phosmet, phosphamidon, phorate, phoxim, pirimiphos-methyl, profenofos, prothiofos, sulprophos, tetrachlorvinphos, terbufos, triazophos, trichlorfon;

A.2. Carbamates: e.g. alanycarb, aldicarb, bendiocarb, benfuracarb, carbaryl, carbofuran, carbosulfan, fenoxycarb, furathiocarb, methiocarb, methomyl, oxamyl, pirimicarb, propoxur, thiodicarb, triazamate;

A.3. Pyrethroids: e.g. allethrin, bifenthrin, cyfluthrin, cyhalothrin, cyphenothrin, cypermethrin, alpha-cypermethrin, beta-cypermethrin, zeta-cypermethrin, deltamethrin, esfenvalerate, etofenprox, fenpropathrin, fenvalerate, imiprothrin, lambda-cyhalothrin, permethrin, prallethrin, pyrethrin I and II, resmethrin, silafluofen, tau-fluvalinate, tefluthrin, tetramethrin, tralomethrin, transfluthrin, profluthrin, dimefluthrin;

A.4. Growth regulators: a) chitin synthesis inhibitors: e.g. benzoylureas: chlorfluazuron, diflubenzuron, flucycloxuron, flufenoxuron, hexaflumuron, lufenuron, novaluron, teflubenzuron, triflumuron; buprofezin, diofenolan, hexythiazox, etoxazole, clofentazine; b) ecdysone antagonists: e.g. halofenozide, methoxyfenozide, tebufenozide, azadirachtin; c) juvenoids: e.g. pyriproxyfen, methoprene, fenoxycarb; d) lipid biosynthesis inhibitors: e.g. spirodiclofen, spiromesifen or spirotetramat;

A.5. Nicotinic receptor agonists/antagonists compounds (nicotinoid insecticides or neonicotinoids): e.g. clothianidin, dinotefuran, imidacloprid, thiamethoxam, nitenpyram, acetamiprid, thiacloprid or the thiazol compound of formula P1

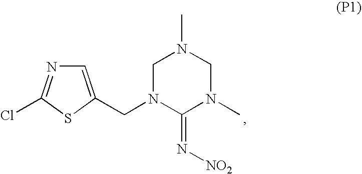

A.6. GABA antagonist compounds: e.g. acetoprole, endosulfan, ethiprole, fipronil, vaniliprole, pyrafluprole, pyriprole, 5-amino-3-(aminothiocarbonyl)-1-(2,6-dichloro-4-trifluoromethyl phenyl)-4-(trifluoromethylsulfinyl)-pyrazole;

A.7. Macrocyclic lactone insecticides: abamectin, emamectin, milbemectin, lepimectin, spinosad,

A.8. Mitochondrial complex I electron transport inhibitors (METI I compounds): e.g. fenazaquin, pyridaben, tebufenpyrad, tolfenpyrad, flufenerim;

A.9. Mitochondrial complex II and/or complex III electron transport inhibitors (METI II and III compounds): e.g. acequinocyl, fluacyprim, hydramethylnon;

A.10. Uncoupler compounds: e.g. chlorfenapyr;

A.11. Oxidative phosphorylation inhibitor compounds: cyhexatin, diafenthiuron, fenbutatin oxide, propargite;

A.12. Moulting disruptor compounds: e.g. cyromazine;

A.13. Mixed function oxidase inhibitor compounds: e.g. piperonyl butoxide;

A.14. Sodium channel blocker compounds: e.g. indoxacarb, metaflumizone,

A.15. Various: benclothiaz, bifenazate, cartap, flonicamid, pyridalyl, pymetrozine, sulfur, thiocyclam, flubendiamide, cyenopyrafen, flupyrazofos, cyflumetofen, amidoflumet, compounds of the formula P2:

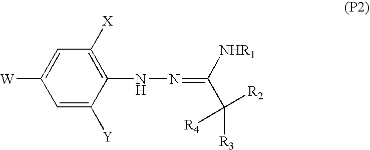

wherein X and Y are each independently halogen, in particular chlorine;
W is halogen or C₁-C₂-haloalkyl, in particular trifluoromethyl;
R₁ is C₁-C₆-alkyl, C₂-C₆-alkenyl, C₂-C₆-alkynyl, C₁-C₄-alkoxy-C₁-C₄-alkyl or C₃-C₆-cycloalkyl each of which may be substituted with 1, 2, 3, 4 or 5 halogen atoms; in particular R¹ is methyl or ethyl;
R₂ and R₃ are C₁-C₆-alkyl, in particular methyl, or may form together with the adjacent carbon atom a C₃-C₆-cycloalkyl moiety, in particular a cyclopropyl moiety, which may carry 1, 2 or 3 halogen atoms, examples including 2,2-dichlorocyclopropyl and 2,2-dibromocyclopropyl; and
R₄ is hydrogen or C₁-C₆-alkyl, in particular hydrogen methyl or ethyl;
   anthranilamide compounds of formula P3

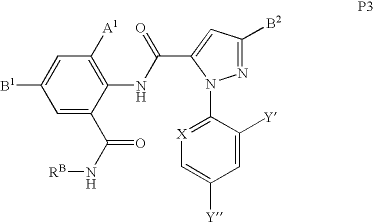

wherein A¹ is CH₃, Cl, Br, I, X is C—H, C—Cl, C—F or N, Y′ is F, Cl, or Br, Y″ is F, Cl, CF₃, B¹ is hydrogen, Cl, Br, I, CN, B² is Cl, Br, CF₃, OCH₂CF₃, OCF₂H, and R^{B} is hydrogen, CH₃ or CH(CH₃)₂;
   and malononitrile compounds as described in JP 2002 284608, WO 02/89579, WO 02/90320, WO 02/90321, WO 04/06677, WO 04/20399, or JP 2004 99597.

Suitable pesticides compounds also include microorganisms such as Bacillus thuringiensis, Bacillus tenebrionis and Bacillus subtilis.

The aforementioned compositions are particularly useful for protecting plants against infestation of said pests and also for protecting plants against infections of phytopathogenic fungi or to combat these pests/fungi in infested/infected plants.

However, the compounds of formula I are also suitable for the treatment of seeds. Application to the seeds is carried out before sowing, either directly on the seeds or after having pregerminated the latter.

Compositions which are useful for seed treatment are e.g.:

A Soluble concentrates (SL, LS)

### D Emulsions (EW, EO, ES)

### E Suspensions (SC, OD, FS)

F Water-dispersible granules and water-soluble granules (WG, SG)
G Water-dispersible powders and water-soluble powders (WP, SP, WS)
H Dustable powders (DP, DS)

Preferred FS formulations of compounds of formula I for seed treatment usually comprise from 0.5 to 80% of the active ingredient, from 0.05 to 5% of a wetter, from 0.5 to 15% of a dispersing agent, from 0.1 to 5% of a thickener, from 5 to 20% of an anti-freeze agent, from 0.1 to 2% of an anti-foam agent, from 1 to 20% of a pigment and/or a dye, from 0 to 15% of a sticker/adhesion agent, from 0 to 75% of a filler/vehicle, and from 0.01 to 1% of a preservative.

Suitable pigments or dyes for seed treatment formulations are pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

Stickers/adhesion agents are added to improve the adhesion of the active materials on the seeds after treatment. Suitable adhesives are block copolymers EO/PO-surfactants but also polyvinylalcohols, polyvinylpyrrolidones, polyacrylates, polymethacrylates, polybutenes, polyisobutylenes, polystyrene, polyethyleneamines, polyethyleneamides, polyethyleneimines (Lupasol®, Polymin®), polyethers and copolymers derived from these polymers.

For use against ants, termites, wasps, flies, mosquitos, crickets, or cockroaches, compounds of formula I are preferably used in a bait composition.

The bait can be a liquid, a solid or a semisolid preparation (e.g. a gel). Solid baits can be formed into various shapes and forms suitable to the respective application e.g. granules, blocks, sticks, disks. Liquid baits can be filled into various devices to ensure proper application, e.g. open containers, spray devices, droplet sources, or evaporation sources. Gels can be based on aqueous or oily matrices and can be formulated to particular necessities in terms of stickyness, moisture retention or aging characteristics. The bait employed in the composition is a product which is sufficiently attractive to incite insects such as ants, termites, wasps, flies, mosquitos, crickets etc. or cockroaches to eat it. The attractiveness can be manipulated by using feeding stimulants or sex pheromones. Food stimulants are chosen, for example, but not exclusively, from animal and/or plant proteins (meat-, fish- or blood meal, insect parts, egg yolk), from fats and oils of animal and/or plant origin, or mono-, oligo- or polyorganosaccharides, especially from sucrose, lactose, fructose, dextrose, glucose, starch, pectin or even molasses or honey. Fresh or decaying parts of fruits, crops, plants, animals, insects or specific parts thereof can also serve as a feeding stimulant. Sex pheromones are known to be more insect specific. Specific pheromones are described in the literature and are known to those skilled in the art.

Formulations of compounds of formula I as aerosols (e.g. in spray cans), oil sprays or pump sprays are highly suitable for the non-professional user for controlling pests such as flies, fleas, ticks, mosquitos or cockroaches. Aerosol recipes are preferably composed of the active compound, solvents such as lower alcohols (e.g. methanol, ethanol, propanol, butanol), ketones (e.g. acetone, methyl ethyl ketone), paraffin hydrocarbons (e.g. kerosenes) having boiling ranges of approximately 50 to 250° C., dimethylformamide, N-methylpyrrolidone, dimethyl sulfoxide, aromatic hydrocarbons such as toluene, xylene, water, furthermore auxiliaries such as emulsifiers such as sorbitol monooleate, oleyl ethoxylate having 3-7 mol of ethylene oxide, fatty alcohol ethoxylate, perfume oils such as ethereal oils, esters of medium fatty acids with lower alcohols, aromatic carbonyl compounds, if appropriate stabilizers such as sodium benzoate, amphoteric surfactants, lower epoxides, triethyl orthoformate and, if required, propellants such as propane, butane, nitrogen, compressed air, dimethyl ether, carbon dioxide, nitrous oxide, or mixtures of these gases.

The oil spray formulations differ from the aerosol recipes in that no propellants are used.

The compounds of formula I and its respective compositions can also be used in mosquito and fumigating coils, smoke cartridges, vaporizer plates or long-term vaporizers and also in moth papers, moth pads or other heat-independent vaporizer systems.

The compounds of formula I and its compositions can be used for protecting non-living material, in particular cellulose-based materials such as wooden materials e.g. trees, board fences, sleepers, etc. and buildings such as houses, outhouses, factories, but also construction materials, furniture, leathers, fibers, vinyl articles, electric wires and cables etc. from ants and/or termites, and for controlling ants and termites from doing harm to crops or human being (e.g. when the pests invade into houses and public facilities). The compounds of formula I are applied not only to the surrounding soil surface or into the under-floor soil in order to protect wooden materials but it can also be applied to lumbered articles such as surfaces of the under-floor concrete, alcove posts, beams, plywoods, furniture, etc., wooden articles such as particle boards, half boards, etc. and vinyl articles such as coated electric wires, vinyl sheets, heat insulating material such as styrene foams, etc. In case of application against ants doing harm to crops or human beings, the ant controller of the present invention is applied to the crops or the surrounding soil, or is directly applied to the nest of ants or the like.

In the methods according to the invention the pests are controlled by contacting the target parasite/pest, its food supply, habitat, breeding ground or its locus with a pesticidally effective amount of at least one compounds I, or the N-oxide or salt thereof, or with a composition, containing a pesticidally effective amount of at least one compound I, or the N-oxide or salt thereof.

“Locus” means a habitat, breeding ground, plant, seed, soil, area, material or environment in which a pest or parasite is growing or may grow.

In general, “pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions such as desired pesticidal effect and duration, weather, target species, locus, mode of application, and the like.

The compounds of the invention can also be applied preventively to places at which occurrence of the pests is expected.

The compounds of formula I may be also used to protect growing plants from attack or infestation by pests by contacting the plant with a pesticidally effective amount of compounds of formula I. As such, “contacting” includes both direct contact (applying the compounds/compositions directly on the pest and/or plant—typically to the foliage, stem or roots of the plant) and indirect contact (applying the compounds/compositions to the locus of the pest and/or plant).

The compounds I are employed by treating the fungi, pests or the plants, seeds, materials or the soil to be protected from fungal attack or pesticidal attack with a fungicidally or pesticidally effective amount of at least one active compound I, its N-oxide or salt. The application can be carried out both before and after the infection/infestation of the materials, plants or seeds by the fungi or pest.

When employed in plant protection, the amounts applied are, depending on the kind of effect desired, in the range of 0.1 g to 4000 g per hectare, desirably from 25 g to 600 g per hectare, more desirably from 50 g to 500 g per hectare.

In the treatment of seed, the application rates of the active compounds are generally from 0.001 g to 100 g per kg of seed, preferably from 0.01 g to 50 g per kg of seed, in particular from 0.01 g to 2 g per kg of seed.

In the case of soil treatment or of application to the pests dwelling place or nest, the quantity of active ingredient ranges from 0.0001 to 500 g per 100 m², preferably from 0.001 to 20 g per 100 m².

Customary application rates in the protection of materials are, for example, from 0.01 g to 1000 g of active compound per m² treated material, desirably from 0.1 g to 50 g per m².

Insecticidal compositions for use in the impregnation of materials typically contain from 0.001 to 95 weight %, preferably from 0.1 to 45 weight %, and more preferably from 1 to 25 weight % of at least one repellent and/or insecticide.

For use in bait compositions, the typical content of active ingredient is from 0.001 weight % to 15 weight %, desirably from 0.001 weight % to 5% weight % of active compound.

For use in spray compositions, the content of active ingredient is from 0.001 to 80 weights %, preferably from 0.01 to 50 weight % and most preferably from 0.01 to 15 weight %.

When used in the protection of materials or stored products, the amount of active compound applied depends on the kind of application area and on the desired effect. Amounts customarily applied in the protection of materials are, for example, 0.001 g to 2 kg, preferably 0.005 g to 1 kg, of active compound per cubic meter of treated material.

Under outdoor conditions, the active compound application rate for controlling pests is from 0.1 to 2.0, preferably from 0.2 to 1.0, kg/ha.

Various types of oils, wetters, adjuvants, herbicides, fungicides, other pesticides, or bactericides may be added to the active compounds, if appropriate not until immediately prior to use (tank mix). These agents can be admixed with the agents according to the invention in a weight ratio of 1:100 to 100:1, preferably 1:10 to 10:1.

Adjuvants which can be used are in particular organic modified polysiloxanes such as Break Thru S 240®; alcohol alkoxylates such as Atplus 245®, Atplus MBA 1303®, Plurafac LF 300® and Lutensol ON 30®; EO/PO block polymers, z. B. Pluronic RPE 2035® and Genapol B®; alcohol ethoxylates such as Lutensol XP 80®; and dioctyl sulfosuccinate sodium such as Leophen RA®.

### SYNTHESIS EXAMPLES

The procedures described in the synthesis examples below were used to prepare further compounds I by appropriate modification of the starting compounds. The compounds thus obtained are listed in the tables below, together with physical data.

### Example 1

### Preparation of 5-bromo-pyridine-2-sulfonic acid picolyl amide

At 0° C., a solution of isopropylmagnesiumchloride (2 M in tetrahydrofuran, 1.1 equivalents (eq.)) was slowly added to 80 mmol of 3-bromo-6-iodo-pyridine in 80 ml of tetrahydrofuran, maintaining the temperature between 0 and 10° C. After stirring for 1 h at about 20° C., the solution was cooled to (−40)° C. Then, 2.5 eq. of SO₂ was added under intense cooling to maintain a temperature of (−40)° C. After 30 minutes at this temperature, 1.1 eq. of SO₂Cl₂ was added carefully. Then, the reaction mixture was warmed to 0° C. After 30 minutes stirring, 10% aqueous hydrochloric acid was added carefully. Then, the crude reaction mixture was extracted with 100 ml of diethyl ether three times. The combined organic phases were washed with saturated aqueous sodium chloride and then dried over sodium sulfate. The solvent was removed and the crude sulfochloride was dissolved in 40 ml of acetonitrile.

Meanwhile, 1.1 equivalent of picolylamine and 1.1 equivalent of triethylamine were dissolved in 50 ml of methylcyanide and cooled to 0° C. The crude sulfochloride in methylcyanide was added via a dropping funnel maintaining the temperature below 10° C. The solution was warmed to about 20° C. and stirred over night. Then, the precipitated solid was filtered off and washed with 30 ml of water. The product obtained was an off-white solid. Yield: 20.0 g (82%); m.p.: 156° C.

### Example 2

### Preparation of 5-(4-methoxyphenyl)-pyridine-2-sulfonic acid picolyl amide

A solution of 0.4 g (1.2 mmol) bromide from example 1, 0.22 g (1.5 mmol) of 4-methoxybenzene boronic acid, 0.03 g of PdCl₂[P(C₆H₅)₃]₂, 0.020 g of P[C(CH₃)₃]₃*HBF₄ and triethylamine was dissolved in 5 ml of methylcyanide and 2 ml of water. The reaction mixture was refluxed for 2 hours. After chromatographic purification 0.28 g of the title compound were obtained as an off-white solid. M.p.: 172° C.

The compounds of the examples 3 to 132 were prepared in an analogous manner and are listed in table B, table C and table D.

TABLE B

| | |
|---|---|
| | (I-A) |
| 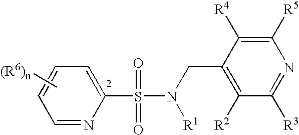 | |

| Examples | R¹ | R² | R³ | R⁴ | R⁵ | (R⁶)ₙ | m.p. |
|---|---|---|---|---|---|---|---|
| Ex. 1 | H | H | H | H | H | 5-Br | 156° C. |
| Ex. 2 | H | H | H | H | H | 5-(4-OCH₃-phenyl) | 172° C. |
| Ex. 3 | H | H | H | H | H | 5-[4-(n-C₃H₇)-phenyl] | 184° C. |
| Ex. 4 | H | H | H | H | H | 5-(4-C₂H₅-phenyl] | 160-162° C. |
| Ex. 5 | H | H | H | H | H | 5-(4-F-phenyl) | 200° C. |
| Ex. 6 | H | H | H | H | H | 5-(3-Cl-phenyl) | 178° C. |
| Ex. 7 | H | H | H | H | H | 5-(4-CF₃-phenyl) | 196° C. |
| Ex. 8 | H | H | H | H | H | 5-[4-CH(CH₃)₂-phenyl] | 188° C. |
| Ex. 9 | H | H | H | H | H | 5-(4-OCF₃-phenyl) | 174° C. |
| Ex. 10 | H | H | H | H | H | 5-(4-Cl-phenyl) | 190-192° C. |
| Ex. 11 | H | H | H | H | H | 5-[4-(CO—CH₃)-phenyl] | 198-200° C. |
| Ex. 12 | H | H | H | H | H | 5-[4-(C(CH₃)═NOCH₃)-phenyl] | |
| Ex. 13 | H | H | H | H | H | 5-[4-(C(CH₃)═NOC₂H₅)-phenyl] | 208-210° C. |
| Ex. 14 | H | H | H | H | H | 5-(3-F, 4-F-phenyl) | 180-182° C. |
| Ex. 15 | H | H | H | H | H | 5-(4-CN-phenyl) | 220° C. |
| Ex. 16 | H | H | H | H | H | 5-(3-CN-phenyl) | |
| Ex. 17 | H | H | H | H | H | 5-(3-F, 4-F-phenyl) | |
| Ex. 18 | H | H | H | H | H | 5-(3-Cl, 4-Cl-phenyl) | 170-172° C. |
| Ex. 19 | H | H | H | H | H | 5-[3-Cl, 4-(OCH₃-phenyl] | 150-152° C. |
| Ex. 20 | H | H | H | H | H | 5-(2-Cl-phenyl) | 65° C. |
| Ex. 21 | H | H | H | H | H | 5-[3,4-(O—CH₂—O)-phenyl] | 178-180° C. |
| Ex. 22 | H | H | H | H | H | 5-(3-Cl, 4-F-phenyl) | 202-205° C. |
| Ex. 23 | H | H | H | H | H | 5-(3-CN-phenyl) | 168-170° C. |
| Ex. 24 | H | H | H | H | H | 5-(2-CH₃, 4-F-phenyl) | 139-140° C. |
| Ex. 25 | H | H | H | H | H | 5-(4-CH₃-phenyl) | 188° C. |
| Ex. 26 | H | H | H | H | H | 5-(2-CH₃-phenyl) | 128° C. |
| Ex. 27 | H | H | H | H | H | 5-(3-CH₃-phenyl) | 151° C. |
| Ex. 28 | H | H | H | H | H | 5-(3-F-phenyl) | 154° C. |
| Ex. 29 | H | H | H | H | H | 5-(2-F-phenyl) | 140° C. |
| Ex. 30 | H | H | H | H | H | 5-(3-CF₃-phenyl) | 167° C. |
| Ex. 31 | H | H | H | H | H | 5-(3-OCH₃-phenyl) | 133° C. |
| Ex. 32 | H | H | H | H | H | 5-(2-OCH₃-phenyl) | 122° C. |
| Ex. 33 | H | H | H | H | H | 5-(2-(CO—NH₂)-phenyl) | 197° C. |
| Ex. 34 | H | H | H | H | H | 5-(2-CF₃-phenyl) | 116° C. |
| Ex. 35 | H | H | H | H | H | 6-[4-(n-C₃H₇)-phenyl] | 170-172° C. |
| Ex. 36 | H | H | H | H | H | 6-(4-C₂H₅-phenyl] | 140-145° C. |
| Ex. 37 | H | H | H | H | H | 6-(4-F-phenyl) | 148° C. |
| Ex. 38 | H | H | H | H | H | 6-(3-Cl-phenyl) | 138° C. |
| Ex. 39 | H | H | H | H | H | 6-(4-CF₃-phenyl) | 143° C. |
| Ex. 40 | H | H | H | H | H | 6-[4-CH(CH₃)₂-phenyl] | 150-152° C. |
| Ex. 41 | H | H | H | H | H | 6-(4-OCF₃-phenyl) | 130-133° C. |
| Ex. 42 | H | H | H | H | H | 6-(4-Cl-phenyl) | 158-160° C. |
| Ex. 43 | H | H | H | H | H | 6-[4-(CO—CH₃)-phenyl] | 138-140° C. |
| Ex. 44 | H | H | H | H | H | 6-[4-(C(CH₃)═NOCH₃)-phenyl] | 130° C. |
| Ex. 45 | H | H | H | H | H | 6-[4-(C(CH₃)═NOC₂H₅)-phenyl] | 170-172° C. |
| Ex. 46 | H | H | H | H | H | 6-(3-Cl, 4-(OCH₃-phenyl) | 165-167° C. |
| Ex. 47 | H | H | H | H | H | 6-[3,4-(O—CH₂—O)-phenyl] | 194-196° C. |
| Ex. 48 | H | H | H | H | H | 6-(2-Cl-phenyl) | 150-153° C. |
| Ex. 49 | H | H | H | H | H | 6-(3-Cl, 4-F-phenyl) | 181-183° C. |
| Ex. 50 | H | H | H | H | H | 6-(4-CN-phenyl) | 210-213° C. |
| Ex. 51 | H | H | H | H | H | 6-(3-CN-phenyl) | 172-174° C. |
| Ex. 52 | H | H | H | H | H | 6-(3-F, 4-F-phenyl) | 155-160° C. |
| Ex. 53 | H | H | H | H | H | 6-(3-Cl, 4-Cl-phenyl) | 180-185° C. |
| Ex. 54 | H | H | H | H | H | 6-[2-CH₃, 4-F-phenyl] | 130-132° C. |
| Ex. 55 | H | H | H | H | H | 6-(4-CH₃-phenyl) | 181° C. |
| Ex. 56 | H | H | H | H | H | 6-(2-CH₃-phenyl) | 169° C. |
| Ex. 57 | H | H | H | H | H | 6-(3-CH₃-phenyl) | 140° C. |
| Ex. 58 | H | H | H | H | H | 6-(3-F-phenyl) | 148° C. |
| Ex. 59 | H | H | H | H | H | 6-(2-F-phenyl) | 151° C. |
| Ex. 60 | H | H | H | H | H | 6-(3-CF₃-phenyl) | 124° C. |
| Ex. 61 | H | H | H | H | H | 6-(4-OCH₃-phenyl) | 169° C. |
| Ex. 62 | H | H | H | H | H | 6-(3-OCH₃-phenyl) | 152° C. |
| Ex. 63 | H | H | H | H | H | 6-(2-OCH₃-phenyl) | 156° C. |
| Ex. 64 | H | H | H | H | H | 6-(2-CN-phenyl) | 122° C. |

m.p. melting point

TABLE C

| | |
|---|---|
| | I-B |
| 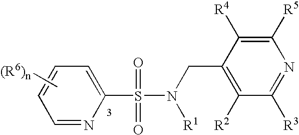 | |

| Example | R¹ | R² | R³ | R⁴ | R⁵ | (R⁶)ₙ | m.p. |
|---|---|---|---|---|---|---|---|
| Ex. 65 | H | H | H | H | H | 6-[4-(n-C₃H₇)-phenyl] | 182° C. |
| Ex. 66 | H | H | H | H | H | 6-(4-C₂H₅-phenyl] | 185° C. |
| Ex. 67 | H | H | H | H | H | 6-(4-F-phenyl) | 180-185° C. |
| Ex. 68 | H | H | H | H | H | 6-(3-Cl-phenyl) | 150° C. |
| Ex. 69 | H | H | H | H | H | 6-(4-CF₃-phenyl) | 210° C. |
| Ex. 70 | H | H | H | H | H | 6-[4-CH(CH₃)₂-phenyl] | 172-176° C. |
| Ex. 71 | H | H | H | H | H | 6-(4-OCF₃-phenyl) | 192-195° C. |
| Ex. 72 | H | H | H | H | H | 6-(4-Cl-phenyl) | 205-210° C. |
| Ex. 73 | H | H | H | H | H | 6-[4-(CO—CH₃-phenyl] | 215-217° C. |
| Ex. 74 | H | H | H | H | H | 6-[4-(C(CH₃)═NOCH₃-phenyl] | 190-192° C. |
| Ex. 75 | H | H | H | H | H | 6-[4-(C(CH₃)═NOC₂H₅)-phenyl] | 167-170° C. |
| Ex. 76 | H | H | H | H | H | 6-[3,4-(O—CH₂—O)-phenyl] | 185-190° C. |
| Ex. 77 | H | H | H | H | H | 6-(2-Cl-phenyl) | 120-122° C. |
| Ex. 78 | H | H | H | H | H | 6-(3-Cl, 4-F-phenyl) | 179-181° C. |
| Ex. 79 | H | H | H | H | H | 6-(4-CN-phenyl) | 200-202° C. |
| Ex. 80 | H | H | H | H | H | 6-(3-CN-phenyl) | 163-165° C. |
| Ex. 81 | H | H | H | H | H | 6-(3-F, 4-F-phenyl) | 180-182° C. |
| Ex. 82 | H | H | H | H | H | 6-(3-Cl, 4-Cl-phenyl) | 183-186° C. |
| Ex. 83 | H | H | H | H | H | 6-[3-Cl, 4-(OCH₃)-phenyl] | 202-204° C. |
| Ex. 84 | H | H | H | H | H | 6-(2-CH₃, 4-F-phenyl) | 153-154° C. |
| Ex. 85 | H | H | H | H | H | 6-(4-CH₃-phenyl) | 230° C. |
| Ex. 86 | H | H | H | H | H | 6-(2-CH₃-phenyl) | 157° C. |
| Ex. 87 | H | H | H | H | H | 6-(3-CH₃-phenyl) | 168° C. |
| Ex. 88 | H | H | H | H | H | 6-(3-F-phenyl) | 173° C. |
| Ex. 89 | H | H | H | H | H | 6-(2-F-phenyl) | 166° C. |
| Ex. 90 | H | H | H | H | H | 6-(3-CF₃-phenyl) | 181° C. |
| Ex. 91 | H | H | H | H | H | 6-(4-OCH₃-phenyl) | 172° C. |
| Ex. 92 | H | H | H | H | H | 6-(3-OCH₃-phenyl) | 128° C. |
| Ex. 93 | H | H | H | H | H | 6-(4-CF₃-phenyl) | |
| Ex. 94 | H | H | H | H | H | 6-(2-(CO—NH₂)-phenyl) | 135° C. |
| Ex. 95 | H | H | H | H | H | 6-[5-(2-CF₃-phenyl)pyridin-3-yl] | |
| Ex. 96 | H | H | H | H | H | 6-[5-(4-CF₃-phenyl)pyridin-3-yl] | |
| Ex. 97 | H | H | H | H | H | 6-(2-CF₃-phenyl) | 105° C. |
| Ex. 98 | H | H | H | H | H | 6-(4-OCH₃-phenyl) | |
| Ex. 99 | H | H | H | H | H | 6-(2-OCH₃-phenyl) | 163° C. |
| Ex. 100 | H | H | H | H | H | 5-[4-(n-C₃H₇)-phenyl] | 138-140° C. |
| Ex. 101 | H | H | H | H | H | 5-(4-C₂H₅-phenyl] | 124° C. |
| Ex. 102 | H | H | H | H | H | 5-(4-F-phenyl) | 186° C. |
| Ex. 103 | H | H | H | H | H | 5-(3-Cl-phenyl) | 145° C. |
| Ex. 104 | H | H | H | H | H | 5-(4-CF₃-phenyl) | 177° C. |
| Ex. 105 | H | H | H | H | H | 5-[4-CH(CH₃)₂-phenyl] | 156° C. |
| Ex. 106 | H | H | H | H | H | 5-(4-OCF₃-phenyl) | 137° C. |
| Ex. 107 | H | H | H | H | H | 5-(4-Cl-phenyl) | 192° C. |
| Ex. 108 | H | H | H | H | H | 5-[4-(CO—CH₃)-phenyl] | 170-172° C. |
| Ex. 109 | H | H | H | H | H | 5-(3-F, 4-F-phenyl) | 201-203° C. |
| Ex. 110 | H | H | H | H | H | 5-(4-CN-phenyl) | 215° C. |
| Ex. 111 | H | H | H | H | H | 5-(2-Cl-phenyl) | 120-122° C. |
| Ex. 112 | H | H | H | H | H | 5-[4-(C(CH₃)═NOCH₃)-phenyl] | 158-160° C. |
| Ex. 113 | H | H | H | H | H | 5-[4-(C(CH₃)═NOC₂H₅)-phenyl] | 173-175° C. |
| Ex. 114 | H | H | H | H | H | 5-(3-Cl, 4-Cl-phenyl) | 205-207° C. |
| Ex. 115 | H | H | H | H | H | 5-[3-Cl, 4-(OCH₃)-phenyl] | 133-137° C. |
| Ex. 116 | H | H | H | H | H | 5-[3,4-(O—CH₂—O)-phenyl] | 178-180° C. |
| Ex. 117 | H | H | H | H | H | 5-(3-Cl, 4-F-phenyl) | 192-195° C. |
| Ex. 118 | H | H | H | H | H | 5-(3-CN-phenyl) | 200-202° C. |
| Ex. 119 | H | H | H | H | H | 5-(2-CH₃, 4-F-phenyl) | 175-177° C. |
| Ex. 120 | H | H | H | H | H | 5-(C═C—CH₂CH₂—C₂H₅) | 92-94° C. |
| Ex. 121 | H | H | H | H | H | 5-(4-CH₃-phenyl) | 204° C. |
| Ex. 122 | H | H | H | H | H | 5-(2-CH₃-phenyl) | 163° C. |
| Ex. 123 | H | H | H | H | H | 5-(3-CH₃-phenyl) | 164° C. |
| Ex. 124 | H | H | H | H | H | 5-(3-F-phenyl) | 180° C. |
| Ex. 125 | H | H | H | H | H | 5-(2-F-phenyl) | 143° C. |
| Ex. 126 | H | H | H | H | H | 5-(3-CF₃-phenyl) | 170° C. |
| Ex. 127 | H | H | H | H | H | 5-(4-OCH₃-phenyl) | 148° C. |
| Ex. 128 | H | H | H | H | H | 5-(3-OCH₃-phenyl) | 151° C. |
| Ex. 129 | H | H | H | H | H | 5-(2-OCH₃-phenyl) | 162° C. |
| Ex. 130 | H | H | H | H | H | 5-(2-(CO—NH₂)-phenyl) | 200° C. |

m.p. melting point

TABLE D

| | |
|---|---|
| | (I-C) |
| 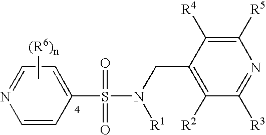 | |

| Ex- | | | | | | | |
|---|---|---|---|---|---|---|---|
| am- | | | | | | | |
| ple | R¹ | R² | R³ | R⁴ | R⁵ | R⁶ | m.p. |
| Ex. | H | H | H | H | H | 2-[4-CH(CH₃)₂-phenyl] | 110° C. |
| 131 | | | | | | | |
| Ex. | H | H | H | H | H | 2-(4-OCF₃-phenyl) | 185-187° C. |
| 132 | | | | | | | |

m.p. melting point

### Examples of the Action Against Harmful Fungi

The fungicidal action of the compounds of the formula I was demonstrated by the following experiments:

The active compounds were formulated separately or together as a stock solution with 0.25% by weight of active compound in acetone or dimethylsulfoxide. 1% by weight of the emulsifier Uniperol® EL (wetting agent having emulsifying and dispersing action based on ethoxylated alkylphenols) was added to this solution and diluted with water to the desired concentration.

### Use Example 1

### Activity Against Early Blight of Tomatoes Caused by Alternaria solani

Young seedlings of tomato plants were grown in pots. These plants were sprayed to run-off with an aqueous suspension, containing the concentration of active compound stated below. The next day, the treated plants were inoculated with an aqueous spore suspension of Alternaria solani containing 0.17×10⁶ spores per ml. Then the trial plants were immediately transferred to a humid chamber. After 5 days at 20 and 22° C. and a relative humidity close to 100%, the extent of fungal attack on the leaves was visually assessed as % diseased leaf area.

In this test the plants which have been treated with 250 ppm of the active compound from examples 8, 66, 69, 70, 72, 75, 78, 90 and 113, respectively showed an infection of not more than 5% and the plants which have been treated with 250 ppm of active compound from examples 2, 9, 13, 61, 67, 74, 84, 91, 111 and 112 respectively showed an infection of not more than 20%, whereas the untreated plants were 90% infected.

### Use Example 2

### Activity Against Late Blight of Tomatoes Caused by Phytophthora infestans, Protective Treatment

Young seedlings of tomato plants were grown in pots. The plants were sprayed to runoff with an aqueous suspension containing the concentration of active compounds stated below. The next day, the treated plants were infected with an aqueous suspension of sporangia of Phytophthora infestans. After inoculation, the trial plants were immediately transferred to a humid chamber. After six days at 18 to and 20° C. and a relative humidity close to 100%, the extent of fungal attack on the leaves was visually assessed as % diseased area.

In this test the plants which have been treated with 250 ppm of the active compound from examples 5, 7, 10, 19, 21, 66, 67, 68, 69, 70, 75, 78 and 112, respectively showed an infection of not more than 5% and the plants which have been treated with 250 ppm of the active compound from examples 6, 8, 13, 17, 18, 25, 28, 72, 74, 86, 92, 119 and 122 respectively showed an infection of not more than 20%, whereas the untreated plants were 90% infected.

### Use Example 3

### Curative Activity Against Brown Rust of Wheat Caused by Puccinia recondita

Leaves of potted wheat seedlings of the variety “Kanzler” were dusted with spores of brown rust (Puccinia recondita). To ensure the success of the artificial inoculation, the plants were transferred to a humid chamber without light and a high humidity and 20 to 22° C. for 24 hours. The next day, the plants were sprayed to run-off with an aqueous suspension containing the concentration of active compound as described below. The plants were allowed to air-dry. Then, the trial plants were cultivated for 8 days in a greenhouse chamber at approximately 22° C. and a relative humidity between 65 to 70%. The extent of fungal attack was visually assessed as % diseased leaf area.

In this test the plants which have been treated with 250 ppm of the active compound from example 34 showed an infection of not more than 5% and the plants which have been treated with 250 ppm of the active compound of examples 32, 62, 95 and 97 respectively showed an infection of not more than 20%, whereas the untreated plants were 90% infected.

### Use Example 4

### Protective Activity Against Brown Rust of Wheat Caused by Puccinia recondita

Leaves of potted wheat seedlings of the cultivar “Kanzler” were sprayed to run-off with an aqueous suspension, containing the concentration of active ingredient as described below. The next day, the plants were inoculated with spores of brown rust (Puccinia recondita). To ensure the success of artificial inoculation, the plants were transferred to a humid chamber without light and high humidity of 20 to 22° C. for 24 h. Then the trial plants were cultivated for 6 days in a greenhouse chamber at ca. 22° C. and a relative humidity between 65 to 70%. The extent of fungal attack on the leaves was visually assessed as % diseased area.

In this test the plants which have been treated with 250 ppm of the active compound from examples 77 and 82, respectively showed an infection of not more than 20%, whereas the untreated plants were 90% infected.

### Use Example 5

### Curative Activity Against Soy Bean Rust Caused by Phakopsora Pachyrhizi

Leaves of pot-grown soy bean seedlings of the variety “Oxford” were inoculated with spores of Phakopsora pachyrhizi. To ensure the success of the artificial inoculation, the plants were transferred to a humid chamber with a relative humidity of about 95% and 23 to 27° C. for 24 h. The next day the plants were sprayed to run-off with an aqueous suspension, containing the concentration of active ingredient as described below. The plants were allowed to air-dry. Then the trial plants were cultivated for 14 days in a greenhouse chamber at 23 to 27° C. and a relative humidity between 60 and 80%. The extent of fungal attack on the leaves was visually assessed as % diseased leaf area.

In this test the plants which have been treated with 250 ppm of the active compound from examples 28, 29, 58, 59, 88, 89 and 125, respectively showed an infection of not more than 5% and the plants which have been treated with 250 ppm of the active compound from examples 54, 55, 83 and 126 respectively showed an infection of not more than 20%, whereas the untreated plants were 90% infected.

The action of the compounds of the formula I against harmful pests was demonstrated by the following experiments:

1. Activity Against Boll Weevil (Anthonomus grandis)

The active compounds were formulated in 1:3 dimethylsulfoxide:water. 10 to 15 eggs were placed into microtiterplates filled with 2% agar-agar in water and 300 ppm formaline. The eggs were sprayed with 20 μl of the test solution, the plates were sealed with pierced foils and kept at 24-26° C. and 75-85% humidity with a day/night cycle for 3 to 5 days. Mortality was assessed on the basis of the remaining unhatched eggs or larvae on the agar surface and/or quantity and depth of the digging channels caused by the hatched larvae. Tests were replicated 2 times

2. Activity Against Mediterranean Fruitfly (Ceratitis capitata)

The active compounds were formulated in 1:3 DMSO:water. 50 to 80 eggs were placed into microtiterplates filled with 0.5% agar-agar and 14% diet in water. The eggs were sprayed with 5 μl of the test solution, the plates were sealed with pierced foils and kept at 27-29° C. and 75-85% humidity under fluorescent light for 6 days. Mortality was assessed on the basis of the agility of the hatched larvae. Tests were replicated 2 times.

3. Activity Against Tobacco Budworm (Heliothis virescens)

The active compounds were formulated in 1:3 dimethylsulfoxide:water. 15 to 25 eggs were placed into microtiterplates filled with diet. The eggs were sprayed with 10 μl of the test solution, the plates were sealed with pierced foils and kept at 27-29° C. and 75-85% humidity under fluorescent light for 6 days. Mortality was assessed on the basis of the agility and of comparative feeding of the hatched larvae. Tests were replicated 2 times.

4. Activity Against Vetch Aphid (Megoura viciae)

The active compounds were formulated in 1:3 DMSO:water. Bean leaf disks were placed into microtiterplates filled with 0.8% agar-agar and 2.5 ppm OPUS™. The leaf disks were sprayed with 2.5 μl of the test solution and 5 to 8 adult aphids were placed into the microtiterplates which were then closed and kept at 22-24° C. and 35-45% under fluorescent light for 6 days. Mortality was assessed on the basis of vital, reproduced aphids. Tests were replicated 2 times.

5. Activity Against Wheat Aphid (Rhopalosiphum padi)

The active compounds were formulated in 1:3 dimethylsulfoxide:water. Barlay leaf disk were placed into microtiterplates filled with 0.8% agar-agar and 2.5 ppm OPUS™. The leaf disks were sprayed with 2.5 μl of the test solution and 3 to 8 adult aphids were placed into the microtiterplates which were then closed and kept at 22-24° C. and 35-45% humidity under fluorescent light for 5 days. Mortality was assessed on the basis of vital aphids. Tests were replicated 2 times.

1-15. (canceled)

16: A pyridin-4-ylmethyl-amide compound of the general formula I 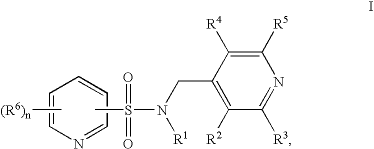
wherein:
R¹ is hydrogen, C₁-C₆-alkyl, C₁-C₆-alkoxy, cyano-C₁-C₄-alkyl, C₁-C₄-haloalkyl, C₁-C₄-alkoxy-C₁-C₄-alkyl, C₁-C₄-haloalkoxy-C₁-C₄-alkyl, di(C₁-C₄-alkyl)amino-C₁-C₄-alkyl, C₃-C₆-cycloalkyl-C₁-C₄-alkyl, C₃-C₆-halocycloalkyl-C₁-C₄-alkyl, (C₁-C₄-alkyl)carbonyl, (C₁-C₄-alkoxy)carbonyl, C₂-C₆-alkenyl, C₃-C₆-cycloalkyl, C₃-C₆-halocycloalkyl, C₅-C₆-cycloalkenyl, saturated 5 or 6-membered N-heterocyclyl-C₁-C₄-alkyl, cyano-C₂-C₄-alkenyl, C₂-C₄-haloalkenyl, C₁-C₄-alkoxy-C₂-C₄-alkenyl, C₁-C₄-haloalkoxy-C₂-C₄-alkenyl, (C₁-C₄-alkyl)carbonyl-C₂-C₄-alkenyl, (C₁-C₄-alkoxy)carbonyl-C₂-C₄-alkenyl, di(C₁-C₄-alkyl)amino-C₂-C₄-alkenyl, C₂-C₆-alkynyl, C₂-C₄-haloalkynyl, C₁-C₄-haloalkyl-C₂-C₄-alkynyl, C₁-C₄-alkoxy-C₂-C₄-alkynyl, tri(C₁-C₄-alkyl)silyl-C₂-C₄-alkynyl, di(C₁-C₄-alkyl)amino, naphthylmethyl or benzyl wherein the last two mentioned radicals may carry at the phenyl or naphthyl ring 1, 2, or 3 radicals, selected from the group consisting of cyano, halogen, C₁-C₄-alkyl, C₁-C₄-haloalkyl, C₁-C₄-alkoxy, C₁-C₄-haloalkoxy, (C₁-C₄-alkyl)carbonyl, (C₁-C₄-alkoxy)carbonyl and di(C₁-C₄-alkyl)amino radical;
R², R³, R⁴, R⁵ independently of one another are selected from the group consisting of hydrogen, halogen, C₁-C₄-alkyl, C₂-C₄-alkenyl, C₂-C₄-alkynyl, tri-C₁-C₄-alkylsilyl, C₁-C₄-haloalkyl, C₁-C₄-alkoxy, C₁-C₄-haloalkoxy, S(O)ₚR¹⁶ and NR¹⁷R¹⁸;
or
R² and R³ together with the carbon atoms to which they are attached, may form a fused 5 or 6-membered carbocycle or a fused 5- or 6-membered heterocycle containing as ring members one, two or three heteroatoms selected from the group consisting of nitrogen, oxygen and sulfur atoms, it being possible for the fused ring to carry one or two radicals R⁷ or R⁸;
R⁶ is halogen, cyano, nitro, C₁-C₁₀-alkyl, C₂-C₁₀-alkenyl, C₂-C₁₀-alkynyl, C₁-C₁₀-alkoxy, C₁-C₁₀-haloalkyl, C₁-C₁₀-haloalkoxy, (C₁-C₄-alkyl)carbonyl, (C₁-C₄-alkoxy)carbonyl, —C(R⁹)═NOR¹⁰, (C₁-C₄-alkyl)aminocarbonyl, di(C₁-C₄-alkyl)aminocarbonyl, 5- or 6-membered heteroaryl or heteroaryloxy, phenyl, or phenoxy, where the phenyl or heteroaryl ring in the last four mentioned radicals may carry one, two or three radicals R¹¹, wherein said heteroaryl and heteroaryloxy radicals contain as ring members one or two heteroatoms selected from the group consisting of nitrogen, oxygen and sulfur atoms;
or two radicals R⁶ together with two adjacent carbon atoms of the pyridyl ring to which they are attached form a fused 5- or 6-membered carbocycle which may be substituted by 1, 2 or 3 radicals R¹²;
R⁷, R⁸ independently of one another are halogen, C₁-C₄-alkyl, C₁-C₄-haloalkyl, C₁-C₄-alkoxy or C₁-C₄-haloalkoxy;
n is 0, 1 or 2;
R⁹ is hydrogen, C₁-C₄-alkyl, C₁-C₄-haloalkyl, C₁-C₄-alkoxy-C₁-C₄-alkyl, C₁-C₄-haloalkoxy-C₁-C₄-alkyl, phenyl which may bear a cyano, halogen, C₁-C₄-alkoxy or C₁-C₄-haloalkoxy radical, or benzyl which may be unsubstituted or substituted with 1, 2 or 3 radicals, selected from the group consisting of from cyano, halogen and C₁-C₄-alkyl;
R¹⁰ is C₁-C₆-alkyl, benzyl, C₂-C₄-alkenyl, C₁-C₄-haloalkyl, C₂-C₄-haloalkenyl, C₂-C₄-alkynyl or C₂-C₄-haloalkynyl;
R¹¹ is nitro, cyano, OH, halogen, C₁-C₄-alkyl, C₁-C₄-haloalkyl, C₁-C₄-alkoxy, C₁-C₄-haloalkoxy, (C₁-C₄-alkoxy)carbonyl, C₁-C₄-alkylcarbonyl, CHO, CO—NH₂, C₁-C₄-alkylaminocarbonyl, di(C₁-C₄-alkyl)aminocarbonyl, C₁-C₄-alkylthio, C₁-C₄-haloalkylthio, C₁-C₄-alkylsulfinyl, C₁-C₄-haloalkylsulfinyl, C₁-C₄-alkylsulfonyl, C₁-C₄-haloalkylsulfonyl, (C₁-C₄-alkyl)amino, di(C₁-C₄-alkyl)amino, tri(C₁-C₄-alkyl)silyl, —C(R³)═NOR¹⁴, C₂-C₄-alkenyl or C₂-C₄-alkynyl;
or two radicals R¹¹ together with two adjacent carbon atoms of the phenyl ring to which they are attached form a fused 5- or 6-membered carbocycle or a fused 5- or 6-membered heterocycle containing as ring members one, two or three heteroatoms selected from the group consisting of nitrogen, oxygen and sulfur atoms, it being possible for the fused ring to carry 1, 2 or 3 radicals R¹
R¹², R^{12a} independently of each other are selected from the group consisting of halogen, cyano, nitro, C₁-C₈-alkyl, C₁-C₈-haloalkyl, C₁-C₈-alkoxy, C₁-C₈-haloalkoxy, (C₁-C₄-alkyl)carbonyl, (C₁-C₄-alkoxy)carbonyl, —C(R^{13a})═NOR¹⁴a, (C₁-C₄-alkyl)aminocarbonyl, di(C₁-C₄-alkyl)aminocarbonyl, phenyl and phenoxy, where the ring in the last two mentioned radicals may carry one, two or three groups R¹⁵;
R¹, R^{13a} independently of each other are selected from the group consisting of hydrogen, C₁-C₄-alkyl, C₁-C₄-haloalkyl, C₁-C₄-alkoxy-C₁-C₄-alkyl, C₁-C₄-haloalkoxy-C₁-C₄-alkyl, phenyl and benzyl which may be unsubstituted or substituted with 1, 2 or 3 radicals, selected from the group consisting of cyano, halogen and C₁-C₄-alkyl, wherein said phenyl may be unsubstituted or substituted with 1, 2 or 3 radicals, selected from the group consisting of cyano, halogen, C₁-C₄-alkoxy and C₁-C₄-haloalkoxy;
R¹⁴, R^{14a} independently of each other are selected from the group consisting of C₁-C₆-alkyl, benzyl, C₂-C₄-alkenyl, C₁-C₄-haloalkyl, C₂-C₄-haloalkenyl, C₂-C₄-alkynyl and C₂-C₄-haloalkynyl;
R¹⁵ is halogen, C₁-C₄-alkyl, C₁-C₄-alkoxy, C₁₋haloalkyl or C₁₋haloalkoxy;
R¹⁶ is C₁-C₄-alkyl or C₁-C₄-haloalkyl and p is 0, 1 or 2; and
R¹⁷, R¹⁸ independently of each other are selected from the group consisting of hydrogen, and C₁-C₆-alkyl, or R¹⁷ and R¹⁸ together with the nitrogen atom to which they are attached form a five- to eight-membered saturated heterocycle which is attached via nitrogen and may contain one, two or three further heteroatoms or heteroatom groups selected from the group consisting of O, N, S, S(O) and S(O)₂ as ring members, it being possible for the heterocycle to carry 1, 2, 3 or 4 substituents selected from the group consisting of C₁-C₄-alkyl, C₁-C₄-haloalkyl and halogen;
and the N-oxide or an agriculturally acceptable salt thereof.

17: The compound of claim 16, wherein each of R², R³, R⁴ and R⁵ is hydrogen.

18: The compound of claim 16, wherein R² and R³, independently of one another, are selected from the group consisting of hydrogen, halogen, C₁-C₄-alkyl, C₂-C₄-alkenyl, C₂-C₄-alkynyl, tri-C₁-C₄-alkylsilyl, C₁-C₄-haloalkyl, C₁-C₄-alkoxy, C₁-C₄-haloalkoxy, S(O)ₚR¹⁶ and NR⁷R⁸, R⁴ and R⁵ are hydrogen, wherein at least one of the radicals R² and R³ is different from hydrogen.

19: The compound of claim 16, wherein n is 1 and R⁶ is 5- or 6-membered heteroaryl or heteroaryloxy containing one or two heteroatoms as ring members, selected from the group consisting of nitrogen, oxygen and sulfur atoms, wherein the heterocycle may be unsubstituted or may carry 1, 2 or 3 radicals R¹¹.

20: A process for the preparation of pyridin-4-ylmethyl-amide compound of claim 16, which comprises reacting a 4-aminomethylpyridine compound of the formula II 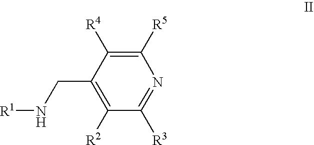
in which R¹ to R⁵ are as defined in claim 1,
under basic conditions with a pyridine sulfonic acid compound of the formula III 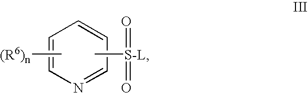
in which R⁶ and n are as defined in claim 1 and L is hydroxy or halogen.

21: An agricultural composition which comprises a solid or liquid carrier and at least one pyridin-4-ylmethyl-amide compound of claim 16, an N-oxide or a salt thereof.

22: The composition of claim 21, wherein in each said compound, N-oxide or salt thereof, independent of one another
(a) each of R², R³, R⁴ and R⁵ is hydrogen;
(b) R² and R³, independently of one another, are selected from the group consisting of hydrogen, halogen, C₁-C₄-alkyl, C₂-C₄-alkenyl, C₂-C₄-alkynyl, tri-C₁-C₄-alkylsilyl, C₁-C₄-haloalkyl, C₁-C₄-alkoxy, C₁-C₄-haloalkoxy, S(O)ₚR¹⁶ and NR¹⁷R¹⁸, R⁴ and R⁵ are hydrogen, wherein at least one of the radicals R² and R³ is different from hydrogen; or
(c) n is 1 and R⁶ is 5- or 6-membered heteroaryl or heteroaryloxy containing one or two heteroatoms as ring members, selected from the group consisting of nitrogen, oxygen and sulfur atoms, wherein the heterocycle may be unsubstituted or may carry 1, 2 or 3 radicals R¹.

23: A method for combating phytopathogenic fungi, which method comprises treating the fungi or the materials, plants, the soil or seeds to be protected against fungal attack, with at least one pyridin-4-ylmethyl-amid compound of the formula I and/or an N-oxide or an agriculturally acceptable salt thereof, according to claim 16.

24: The method of claim 23, wherein in each said compound, N-oxide or salt thereof, independent of one another
(a) each of R², R³, R⁴ and R⁵ is hydrogen;
(b) R² and R³, independently of one another, are selected from the group consisting of hydrogen, halogen, C₁-C₄-alkyl, C₂-C₄-alkenyl, C₂-C₄-alkynyl, tri-C₁-C₄-alkylsilyl, C₁-C₄-haloalkyl, C₁-C₄-alkoxy, C₁-C₄-haloalkoxy, S(O)ₚR¹⁶ and NR¹⁷R¹⁸, R⁴ and R⁵ are hydrogen, wherein at least one of the radicals R² and R³ is different from hydrogen; or
(c) n is 1 and R⁶ is 5- or 6-membered heteroaryl or heteroaryloxy containing one or two heteroatoms as ring members, selected from the group consisting of nitrogen, oxygen and sulfur atoms, wherein the heterocycle may be unsubstituted or may carry 1, 2 or 3 radicals R¹.

25: A method for combating arthropodal pests, which comprises contacting said pests, their habitat, breeding ground, food supply, plant, seed, soil, area, material or environment in which said arthropodal pests are growing or may grow, or the materials, plants, seeds, soils, surfaces or spaces to be protected from an attack of or infestation by said pests, with at least one pyridin-4-ylmethyl-amide compound of claim 16, or with a composition comprising a compound of claim 1.

26: The method of claim 25, wherein in each said compound, N-oxide or salt thereof, independent of one another
(a) each of R², R³, R⁴ and R⁵ is hydrogen;
(b) R² and R³, independently of one another, are selected from the group consisting of hydrogen, halogen, C₁-C₄-alkyl, C₂-C₄-alkenyl, C₂-C₄-alkynyl, tri-C₁-C₄-alkylsilyl, C₁-C₄-haloalkyl, C₁-C₄-alkoxy, C₁-C₄-haloalkoxy, S(O)ₚR¹⁶ and NR¹⁷R¹⁸, R⁴ and R⁵ are hydrogen, wherein at least one of the radicals R² and R³ is different from hydrogen; or
(c) n is 1 and R⁶ is 5- or 6-membered heteroaryl or heteroaryloxy containing one or two heteroatoms as ring members, selected from the group consisting of nitrogen, oxygen and sulfur atoms, wherein the heterocycle may be unsubstituted or may carry 1, 2 or 3 radicals R¹¹.

27: A method for protecting crops from attack or infestation by arthropodal pests, the method comprising contacting a crop with a compound of claim 16.

28: The method of claim 27, wherein in each said compound, N-oxide or salt thereof, independent of one another
(a) each of R², R³, R⁴ and R⁵ is hydrogen;
(b) R² and R³, independently of one another, are selected from the group consisting of hydrogen, halogen, C₁-C₄-alkyl, C₂-C₄-alkenyl, C₂-C₄-alkynyl, tri-C₁-C₄-alkylsilyl, C₁-C₄-haloalkyl, C₁-C₄-alkoxy, C₁-C₄-haloalkoxy, S(O)ₚR¹⁶ and NR¹⁷R¹⁸, R⁴ and R⁵ are hydrogen, wherein at least one of the radicals R² and R³ is different from hydrogen; or
(c) n is 1 and R⁶ is 5- or 6-membered heteroaryl or heteroaryloxy containing one or two heteroatoms as ring members, selected from the group consisting of nitrogen, oxygen and sulfur atoms, wherein the heterocycle may be unsubstituted or may carry 1, 2 or 3 radicals R¹¹.

29: A method for protecting seed from infestation by arthropodal pests and of the seedlings' roots and shoots from infestation by arthropod pests, the method comprising contacting the seed or of the seedlings' roots and shoots with a compound of claim 16.

30: The method of claim 29, wherein in each said compound, N-oxide or salt thereof, independent of one another
(a) each of R², R³, R⁴ and R⁵ is hydrogen;
(b) R² and R³, independently of one another, are selected from the group consisting of hydrogen, halogen, C₁-C₄-alkyl, C₂-C₄-alkenyl, C₂-C₄-alkynyl, tri-C₁-C₄-alkylsilyl, C₁-C₄-haloalkyl, C₁-C₄-alkoxy, C₁-C₄-haloalkoxy, S(O)ₚR¹⁶ and NR¹⁷R¹⁸, R⁴ and R⁵ are hydrogen, wherein at least one of the radicals R² and R³ is different from hydrogen; or
(c) n is 1 and R⁶ is 5- or 6-membered heteroaryl or heteroaryloxy containing one or two heteroatoms as ring members, selected from the group consisting of nitrogen, oxygen and sulfur atoms, wherein the heterocycle may be unsubstituted or may carry 1, 2 or 3 radicals R¹¹.

31: A method for protecting non-living materials from attack or infestation by arthropodal pests, the method comprising contacting the non-living material with a compound of claim 16.

32: The method of claim 31, wherein in each said compound, N-oxide or salt thereof, independent of one another
(a) each of R², R³, R⁴ and R⁵ is hydrogen;
(b) R² and R³, independently of one another, are selected from the group consisting of hydrogen, halogen, C₁-C₄-alkyl, C₂-C₄-alkenyl, C₂-C₄-alkynyl, tri-C₁-C₄-alkylsilyl, C₁-C₄-haloalkyl, C₁-C₄-alkoxy, C₁-C₄-haloalkoxy, S(O)ₚR¹⁶ and NR¹⁷R¹⁸, R⁴ and R⁵ are hydrogen, wherein at least one of the radicals R² and R³ is different from hydrogen; or
(c) n is 1 and R⁶ is 5- or 6-membered heteroaryl or heteroaryloxy containing one or two heteroatoms as ring members, selected from the group consisting of nitrogen, oxygen and sulfur atoms, wherein the heterocycle may be unsubstituted or may carry 1, 2 or 3 radicals RK 1.

33: Seed comprising a compound of claim 16, in an amount of from 0.1 g to 10 kg per 100 kg of seed.

34: The seed of claim 33, wherein in each said compound, N-oxide or salt thereof, independent of one another
(a) each of R², R³, R⁴ and R⁵ is hydrogen;
(b) R² and R³, independently of one another, are selected from the group consisting of hydrogen, halogen, C₁-C₄-alkyl, C₂-C₄-alkenyl, C₂-C₄-alkynyl, tri-C₁-C₄-alkylsilyl, C₁-C₄-haloalkyl, C₁-C₄-alkoxy, C₁-C₄-haloalkoxy, S(O)ₚR¹⁶ and NR⁷R⁸, R⁴ and R⁵ are hydrogen, wherein at least one of the radicals R² and R³ is different from hydrogen; or
(c) n is 1 and R⁶ is 5- or 6-membered heteroaryl or heteroaryloxy containing one or two heteroatoms as ring members, selected from the group consisting of nitrogen, oxygen and sulfur atoms, wherein the heterocycle may be unsubstituted or may carry 1, 2 or 3 radicals R¹¹.
